(19) United States
(12) Patent Application Publication
(10) Pub. No.: US 2016/0145344 A1
(43) Pub. Date: May 26, 2016

(54) MURINE AND HUMAN INNATE LYMPHOID CELLS AND LUNG INFLAMMATION

(71) Applicant: University of Southern California, Los Angeles, CA (US)

(72) Inventor: Omid Akbari, Santa Monica, CA (US)

(73) Assignee: University of Southern California, Los Angeles, CA (US)

(21) Appl. No.: 14/918,277

(22) Filed: Oct. 20, 2015

Related U.S. Application Data
(60) Provisional application No. 62/066,109, filed on Oct. 20, 2014.

(51) Int. Cl. C07K 16/28

(52) U.S. Cl. CPC C07K 16/2896

(57) ABSTRACT

Described herein are methods and compositions for treatment of inflammation, such as inflammation in lung and/or airway tissue, including asthma Innate lymphoid cells (ILCs), such as type 2 ILC2s, are herein described as capable of IL-33 signaling activation, leading to airway hyperresponsiveness (AHR) and inflammation. Further described is the hereto unknown discovery that ICOS-ligand is expressed in ILC2s, that ICOS binding of ICOS to ICOS-ligand is required for its function in ILC2s, and that while IL-33 treatment induces AHR in control mice, IL-33 cannot induce AHR in mice receiving treatment via anti-ICOS-ligand antibodies. These results suggest new methods and compositions targeting ICOS and ICOS-ligand, such as dual specific antibodies that recognize ICOS and ICOS-ligand, an expression profile unique to ILC2s.

### CROSS-REFERENCE TO RELATED APPLICATION

This application claims the benefit of U.S. Provisional Application No. 62/066,109, filed Oct. 20, 2014.

### STATEMENT REGARDING FEDERALLY-SPONSORED RESEARCH

This invention was made with government support under Grant No. AI066020 awarded by the National Institutes of Health. The government has certain rights in the invention.

### FIELD OF THE INVENTION

Described herein are methods and compositions for treatment of inflammation in lung and/or airway tissue, including asthma by targeting innate lymphoid cells (ILCs), such as type 2 ILC2s, responsible for hyperresponsiveness (AHR) and inflammation.

### BACKGROUND

Asthma is a chronic inflammatory condition with hallmark features of airway inflammation, airway hyperresponsiveness and augmented mucus secretion. Allergic asthma is induced by Th2 cytokines in response to allergen exposure, and it is now well-established that members of the CD28 family of T-cell co-stimulatory molecules are involved in Th2 cell differentiation. In addition to Th2 cells and adaptive immunity, it is becoming increasingly clear that non-Th2 cells are also involved in regulating and shaping the inflammation in asthma. Amongst non-Th2 cells, the recently described innate lymphoid cell type (ILCs) appear to be important cellular actors, including Type 2 innate lymphoid cells (ILC2s) that appear to be innate immunity counterparts to Th2 adaptive immunity cells. However, unlike adaptive immune cells, ILCs lack rearranged antigen-specific receptors, responding instead to innate signals. In the context of asthma, ILC2s are notable for their apparent response to canonical type 2 response cytokine initiator IL-33, and for production of copious amount of IL-5 and IL-13 that induce airway hyperreactivity (AHR), a cardinal feature of asthma.

Of therapeutic interest is deciphering the role of CD28 family members, notable for their role in adaptive immunity cellular response and identifying possible roles in innate immunity, such as that in ILCs. For example, Inducible T-cell COStimulator (ICOS) is an important co-stimulatory molecule in T cell subsets. Studies using ICOS-deficient animals have confirmed a critical role for Th2 cell differentiation, germinal center formation and Th2-mediated antibody class switching. While it is known that ICOS can be constitutively expressed by ILCs such as ILC2s, the role of ICOS in function and homeostasis of ILC2s remains unknown. Described herein is the discovery that ICOS and its interaction with ICOS-Ligand in cytokine production and homeostasis are required for functional response of murine and human ILC2s. Based on the hereto unknown expression by human and murine ILC2s of both ICOS and ICOS-Ligand, knockout and humanized mice studies indicate that ICOS:ICOS-Ligand interaction is crucial for the function and homeostatic survival of ILC2s. The lack of ICOS:ICOS-Lignad interaction alters Signal Transducer and Activator of Transcription 5 (STAT5), and such mechanisms of induction of allergic asthma suggest new therapeutic approaches that target ICOS:ICOS-Ligand pathway in ILCs, as lack of ICOS on ILC2s is shown by the Inventors to significantly reduce AHR and lung inflammation. Based on this discovery that ICOS:ICOS-ligand interaction promotes cytokine production in pulmonary ILC2s, therpauetic efficacy of compositions, such as blocking ICOS:ICOS-ligand interaction via antibody binding or similar means, is capable of reducing lung inflammation and AHR. These results establish that ICOS:ICOS-ligand signaling pathway are critically involved in ILC2 function and homeostasis and administration of compositions targeting this interaction can be used in dampening pulmonary inflammation in asthma.

### SUMMARY OF THE INVENTION

Described herein is method for treating inflammation in lung and/or airway tissue in a subject, including selecting a subject with inflammation in lung and/or airway tissue and administering a quantity of a therapeutic agent, wherein the therapeutic agent treats inflammation in lung and/or airway tissue. In other embodiments, the therapeutic agent includes an antibody capable of binding to ICOS, ICOS ligand, or both, and a pharmaceutically acceptable carrier. In other embodiments, the therapeutic agent includes a composition capable of modulating ICOS expression and a pharmaceutically acceptable carrier. In other embodiments, the therapeutic agent includes a composition capable of modulating ICOS ligand expression and a pharmaceutically acceptable carrier. In other embodiments, the therapeutic agent includes a composition capable of modulating a type 2 inflammatory response, and a pharmaceutically acceptable carrier. In other embodiments, modulating a type 2 inflammatory response, includes a reduction in the expression of IL-33 and/or IL-25. In other embodiments, modulating a type 2 inflammatory response, includes a reduction in the expression of one or more of: IL-4, IL-5, and IL-13. In other embodiments, the lung and/or airway tissue includes bronchiolar and/or aveolar tissue. In other embodiments, the lung and/or airway tissue includes epithelial tissue. In other embodiments, treating inflammation includes a reduction in the number of innate lymphoid cells (ILCs). In other embodiments, the ILC are type 2 ILCs (ILC2) cells.

In other embodiments, the ILC2 cells do not express one or more of: CD3, CD14, CD16, CD19, CD20, CD56, CD235a, CD1a, and CD123. In other embodiments, the ILC2 cells express one or more of: CD45, CRTH2, CD127 and CD161. In other embodiments, treating inflammation includes a reduction in STAT5 pathway activation in ILCs.

Also described herein is a pharmaceutical composition, including: a quantity of a therapeutic agent including a composition capable of binding or modulating expression of ICOS, ICOS ligand, or both; and a pharmaceutically acceptable carrier. In other embodiments, the composition includes an antibody.

Also described herein is a method of modulating inflammation, including: selecting a subject in need of treatment for inflammatory related disease and/or condition; and administering a therapeutic agent to the subject, wherein the administration of the composition modulates inflammation in the subject. In other embodiments, the inflammatory related disease and/or condition is acute. In other embodiments, the inflammatory related disease and/or condition is chronic. In other embodiments, the inflammatory related disease and/or condition is a lung related disease and/or condition. In other embodiments, modulating inflammation in the subject includes decreased type 2 ILCs (ILC2) cell phenotype. In other embodiments, the ILC2 cells do not express one or more of: CD3, CD14, CD16, CD19, CD20, CD56, CD235a, CD1a, and CD123. In other embodiments, the ILC2 cells express one or more of: CD45, CRTH2, CD127 and CD161

### BRIEF DESCRIPTION OF FIGURES

FIG. 1. ICOS deficient mice exhibit reduced AHR and inflammation in response to intranasal administration of IL-33 and lower number of pulmonary ILC2s. FIG. 1A BALB/cBYJ or ICOS deficient mice were intranasally challenged with recombinant mouse IL-33 (0.5 μs/mouse) or PBS on days 1-3 followed by measurement of lung function and sample withdrawal on day 4. FIG. 1B Lung resistance and FIG. 1C dynamic compliance in response to increasing doses of inhaled methacholine. FIG. 1D Bar graph presentation of total number of eosinophils in bronchoalveolar, FIG. 1E Lung histology, FIG. 1F Flow cytometry analysis of lung ILC2s in PBS treated WT and ICOS^{-/-} mice as defined by lack of expression of lineage markers (CD3e, CD45R, CD11c, CD11b, TER-119, Gr-1, NK1.1, TCR-δγ and FCεRI) and expression of CD90, CD45 and ST2 gated on single cells, FIG. 1G Total number of ILC2s in the lungs of WT and ICOS^{-/-} in PBS and IL-33 treated mice. h) Data are representative of at least 3 independent experiments and bar-graphs shown as mean±SEM (n=5). (**: P<0.01 ICOS^{-/-}+IL33 compared to WT+IL33, *: P<0.05 ICOS^{-/-}+IL33 compared to WT+IL33, ###: P<0.005 WT+IL-33 compared to WT+PBS, ##: P<0.01 WT+IL-33 compared to WT+PBS).

FIG. 2. Lack of ICOS increases cell death and impairs cytokine production in ILC2s. BALB/cBYJ and ICOS deficient mice received i.n. IL-33 (0.5 μs/mouse) or PBS and after 24 hours lungs were either immediately analyzed for apoptosis, cell death and proliferation or cultured in the presence of IL-33 (20 ng/ml) and Berfeldin A (1 μg/ml) for 6 hours followed by intracellular cytokine analysis. Pulmonary ILC2s were gated based on lineage-, CD45⁺, CD90.2⁺, ST2⁺ and CD25⁺. FIG. 2A Dot plot presentation of flow cytometry analysis of cell death and Annexin V binding of pulmonary ILC2 cells of WT and ICOS^{-/-} mice. EA: early apoptotic (Annexin V⁺, DCD⁻), LA: late apoptotic (Annexin V⁺, DCD^{int}) and dead cells (DCD^{hi}). Numbers show the percentage of the gate, FIG. 2B The frequency of E.A., L.A. and dead cells within ILC2s as determined by flow cytomtery. FIG. 2C histogram demonstration of expression of Ki-67 in lung ILC2 cells as an indication of proliferation. FIG. 2D Mean fluorescence intensity of Ki-67 in pulmonary ILC2 cells. FIG. 2E Dot-plots demonstrate the level of IL-5, IL-13, IL-4 and IL-17A in pulmonary ILC2 cells of WT and ICOS^{-/-} mice. FIG. 2F Alternatively pulmonary ILC2 cells were purified by FACS and cultured (10⁴ cells/100 μl) in the presence of rm-IL-33 (20 ng/ml), rm-IL-2 (10 ng/ml) and rm-IL-7 (10 ng/ml) for 24 and 48 hours. The level of IL-5 and IL-13 produced by purified lung ILC2 cells as measured by ELISA. Data are representative of at least 3 independent experiments and shown as mean±SEM (n=5, **: P<0.01, *: P<0.05).

FIG. 3. Lack of ICOS impairs STAT5 signaling in pulmonary ILC2s. FIG. 3A Histograms show the level of expression of ICOS, CD25, CD127, ST2, CD117, and Sca-1 in WT (thin line) and ICOS^{-/-} (thick line) 24 hours after intranasal administration of PBS (upper panels) or IL-33 (0.5 μs/mouse, Lower panels). The level of isotype-matched stain control is shown as gray filled histogram. FIG. 3B Histogram demonstration (left panel) and median fluorescence intensity (right panel) of phosphorylated STAT5 in pulmonary ILC2s. FIG. 3C Histogram demonstration (left panel) and median fluorescence intensity (right panel) of GATA3 expression in pulmonary ILC2s. Data are representative of at least three independent experiments and are presented as mean±SEM (n=3-4, **: P<0.01, *: P<0.05).

FIG. 4. ICOS deficient ILC2 cells fail to induce airway hyperreactivity and inflammation. FIG. 4A ILC2 cells were purified from BALB/cBYJ and ICOS^{-/-} mice using FACS then injected into RAG2^{-/-}GC^{-/-} mice intravenously (1.5×10⁴ cells/mouse) followed by three intranasal challenges with rm-IL-33 (0.5 μs/mouse) or PBS on three consecutive days. One day after the last challenge lung function was measured and samples were collected. FIG. 4B Lung resistance and FIG. 4C dynamic compliance to increasing doses of methacholine. FIG. 4D Histology of lungs of wild type versus ICOS^{-/-} mice after PBS or IL-33 treatment. Data are representative of at least 3 independent experiments and shown as mean±SEM (n=3). (**: P<0.01 FIG. 4E ICOS^{-/-}+IL33 compared to WT+IL33 and WT-IL33 compared to WT-PBS, *: P<0.05 ICOS^{-/-}+IL33 compared to WT+IL33 and WT-IL33 compared to WT-PBS).

FIG. 5. Blocking ICOS inhibits airway hyperreactivity and lung inflammation in RAG2 deficient mice. FIG. 5A RAG2^{-/-} mice received 500 μs/mouse anti-mouse ICOS blocking antibody or rat IgG2b (isotype control) on day 1 and received rm-IL-33 (0.5 μs/mouse) or PBS intranasally on day 1 to 3 followed by measurement of lung function, performing bronchoalveolar and lung histology on day 4. FIG. 5B Lung resistance and FIG. 5C dynamic compliance in response to increasing doses of methacholine. FIG. 5D Lung histology. FIG. 5E Total number of eosinophils in bronchoalveolar. FIG. 5F Total number and FIG. 5G frequency of pulmonary ILC2 cells as determined by flow cytometry. FIG. 511 Dot plot demonstration of intracellular cytokine production by lung ILC2 cells after 4 hour of culture in the presence of Berfeldin A (1 μg/ml). FIG. 5I Median fluorescence intensity of intracellular IL-5 and IL-13 in lung ILC2 cells. Data are representative of at least three independent experiments and are presented as mean±SEM (n=5) (*: P<0.05 anti-ICOS+IL33 compared to isotype+IL33, ###: P<0.005 isotype+IL-33 compared to isotype+PBS, ##: P<0.01 isotype+IL-33 compared to isotype+PBS).

FIG. 6. Blocking ICOS inhibits Alternaria-induced airway hyperreactivity and lung inflammation. FIG. 6A RAG2^{-/-} mice received intraperitoneal injection of anti-mouse ICOS blocking antibody (500 μs/mouse) or rat IgG2b (isotype control) on day 1 and received extract of Alternaria alternata (100 μs/mouse) or PBS intranasally on day 1 to 4 followed by measurement of lung function, performing BAL and lung histology on day 5. FIG. 6B Lung resistance and FIG. 6C dynamic compliance in response to increasing doses of methacholine. FIG. 6D Lung histology, FIG. 6E Total number of eosinophils in bronchoalveolar. FIG. 6F Total number of lung ILC2 cells. Data are representative of at least three independent experiments and are presented as mean±SEM (n=4, **: P<0.01, *: P<0.05).

FIG. 7. Murine pulmonary ILC2s express ICOS-ligand. FIG. 7A histogram presentation of expression of ICOS-ligand by pulmonary ILC2 cells in BALB/cBYJ (thin line) and in ICOS deficient (thick line) mice in steady state (left panel) and 24 hours after intranasal IL-33 (0.5 μs/mouse) stimulation. The level of isotype-matched stain control is shown as gray filled histogram. FIG. 7B Median fluorescence intensity of ICOS-ligand in wild type and ICOS deficient mice in steady state and after intranasal IL-33 stimulation. FIG. 7C histogram of expression of ICOS-ligand by lung ILC2 cells in BALB/cBYJ mice 24 hours after administration of blocking anti-ICOS (thick line) or rat IgG2a (thin line) in PBS or IL-33 treated mice. FIG. 7D Median fluorescence intensity of ICOS-ligand in lung ILC2 cells of BALB/cBYJ mice treated with blocking anti-ICOS (black bars) or rat IgG2a isotype control (white bars) antibody after PBS or IL-33 administration. FIG. 7E Histogram of the level of phosphorylated STAT5 24 hours after in vitro culture of purified ILC2s in the present of plate-bound ICOS-ligand IgG or human-IgG as isotype control. FIG. 7F Production of IL-13 by purified ILC2s (10⁴/100 μl) after 24 hours culture in the present of plate-bound ICOS-ligand IgG or human-IgG and rm-IL-2 (20 ng/ml), rm-IL-7 (20 ng/ml) and rm-IL-33 (20 ng/ml) as measured by ELISA. Data are representative of at least three independent experiments and are presented as mean±SEM (n=3-4, **: P<0.01, *: P<0.05).

FIG. 8. Human peripheral ILC2 cells express ICOS and ICOS-ligand and blocking their interaction reduces cytokine production by ILC2 cells. Human peripheral blood mononuclear cells were isolated using Ficoll based gradient isolation and cultured in the presence or absence of recombinant human IL-33 (20 ng/ml), IL-2 (10 ng/ml) and IL-7 (20 ng/ml) for 24 hours. FIG. 8A Human peripheral ILC2s were gated on single cells, CD45⁺ CRTH2⁺ Lineage⁻ (CD3, CD14, CD16, CD19, CD20, CD56, CD235a, CD1a, CD123), CD127⁺ and CD161⁺ cells, FIG. 8B Expression of ICOS and FIG. 8C ICOS-ligand by human peripheral ILC2 in freshly isolated (left panels), or cultured in the absence or presence of rh-IL-33 (20 ng/ml, middle and right panels) for 24 hours. Stain isotype control is shown in gray filled histogram. FIG. 8D Human ILC2 cells were purified from PBMCs using FACS and cultured (10⁴/ml) in the presence of rhuman-IL-33 (20 ng/ml), IL-2 (10 ng/ml) and IL-7 (20 ng/ml) for 24 hours. Data are representative of 4 individual donors. FIG. 8E Human peripheral ILC2s were purified using FACS, cultured with rh-IL2 (20 ng/ml) and rh-IL-7 (20 ng/ml) for 48 hours then adoptively transferred into 2 group of RAG2^{-/-} GC^{-/-} mice receiving either anti-human and anti-mouse ICOS-ligand (500 μs/mouse) or isotype control (500 μs/mouse) on day 1. Both groups received either rh-IL-33 (0.5 μg/mouse) or PBS i.n. on day 1-3 followed by dissection on day 4. FIG. 8F Lung resistance of mice in response to increasing doses of methacholine. FIG. 8G Total number of eosinophils in BAL. FIG. 8H Total number of human ILC2s in the lungs of humanized mice (n=3-4).

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As described, allergic asthma is induced by Th2 cytokines in response to allergen exposure. The chronic inflammatory state of airways that is the molecular and physiological hallmark of the disease is initiated by type 2 immune response including Th2 cytokines such as IL-4, IL-5 and IL-13. Among these cytokines, IL-4 is crucial in IgE production by B cells and differentiation of Th2 cells while IL-5 plays an important role in activation and recruitment of eosinophils to the airways, the site of allergen exposure. IL-13 cause goblet cells hyperplasia and increased mucus production while IL-13 alone increases the sensitivity of airway smooth muscle cells to stimuli and leads to airway hyperreactivity (AHR), a cardinal feature of asthma. Initially, it was thought that production of Th2 cytokines only require adaptive immunity, however it has become apparent that innate lymphoid cells (ILCs), the newly discovered subset of lymphoid cells presenting a parallel universe to what has been discovered for adaptive immunity processes in asthma. These cells can also rapidly produce large amounts of Th2 cytokines independent of adaptive immunity. For example, cells such as type 2 innate lymphoid cells (ILC2s, also known as NHCs, nuocytes) respond to IL-33 and produce copious amount of IL-5 and IL-13 that induce airway hyperreactivity (AHR), a cardinal feature of asthma.

The discovery of type 2 ILCs (ILC2s) originated with the observations that intranasal administration of canonical type 2 cytokine initiator IL-25 can lead to production of IL-5 and IL-13 in the lungs of recombination activating gene (RAG) deficient mice, despite lacking mature B and T cells. Suggesting the existence of a non-Th2 cell type, ILC2s were later discovered in the lungs of human and mouse and characterized as the cells causing allergic lung inflammation. In a murine model of allergic asthma besides Th2 cells, ILC2s have been identified as a major source of IL-5 and IL-13. ILC2s are responsive to IL-25, IL-33, Thymic stromal lymphopoietin (TSLP) and by fungal allergens such as Alternaria. It has been shown that IL-33 is more potent than IL-25 in activating ILC2s, and these cells also play a role in maintaining airway epithelial integrity through production of amphiregulin. ILC2s have been shown to express IL2Ra (CD25), IL-7Ra (CD127), IL-33R (T1/ST2), c-Kit (CD117), Sca-1 besides CD45 and CD90 and lack of expression of lineage markers in mice. Human ILC2s are defined based on the expression of CRTH2, CD127 and CD161 besides expression of CD45 and lack of expression of lineage markers. ILC2s do not express recombination-activating gene and unlike T or B cells act in non-antigen specific manner.

Interestingly, murine ILC2s express high levels of Inducible T-cell COStimulator (ICOS), the CD28 family member that is an important co-stimulatory molecule in T cell subsets. CD28 family members contain a single immunoglobulin V-like domain, and ICOS is the third member of the family with notable differences. For example, CD28 and CTLA-4 have a MYPPPY motif that is essential for binding B7-1 and B7-2, whereas ICOS has a FDPPPF motif and capable of binding its ligand, ICOS-ligand, but not B7-1 and B7-2. ICOS was first identified as an inducible T cells co-stimulator related to CD28 superfamily and is highly expressed on tonsilar T cells. It was later shown that ICOS is expressed by activated as well as regulatory T cells and is crucial for T cells survival and function, Th2 differentiation and for lung inflammatory response. Upon binding ICOS to ICOS-ligand a cascade of intracellular signaling molecules are activated that prevent apoptosis and lead to the production of cytokines such as IL-4 and IL-13. ICOS is a costimulatory is constitutively expressed by ILC2s, but to date, ICOS-ligand has been reported to be expressed by B cells, non-lymphoid and lung epithelial cells, but not by T cells or innate lymphoid cells. Further, it is suggested that ICOS-Ligand is down-regulated upon binding to ICOS, providing a possible immunoregulatory mechanism.

However, the functional requirement of ICOS for the function and survival of ILC2 remains totally known as must be elucidated. However, the role of ICOS in function and homeostasis of ILC2s remains unknown. Here the Inventors show that lack of ICOS on ILC2s, significantly reduce AHR and lung inflammation. ICOS:ICOS-ligand interaction promotes cytokine production in pulmonary ILC2s. Utilizing ILC2 humanized mice the Inventors show that blocking ICOS:ICOS-ligand interaction reduces lung inflammation and AHR. These studies demonstrate that ICOS:ICOS-ligand signaling pathway are critically involved in ILC2 function and homeostasis and thus can be used in dampening pulmonary inflammation in asthma.

Described herein are methods for treating inflammation in lung and/or airway tissue in a subject, including selecting a subject with inflammation in lung and/or airway tissue and administering a quantity of a therapeutic agent, wherein the therapeutic agent treats inflammation in lung and/or airway tissue. In various embodiments, the therapeutic agent includes an antibody capable of binding to ICOS, ICOS ligand, or both, and a pharmaceutically acceptable carrier. In various embodiments, the therapeutic agent includes a composition capable of modulating ICOS expression and a pharmaceutically acceptable carrier. In various embodiments, the therapeutic agent includes a composition capable of modulating ICOS ligand expression and a pharmaceutically acceptable carrier. In various embodiments, the therapeutic agent includes a composition capable of modulating a type 2 inflammatory response, and a pharmaceutically acceptable carrier. In various embodiments, modulating a type 2 inflammatory response, includes a reduction in the expression of IL-33 and/or IL-25. In various embodiments, modulating a type 2 inflammatory response, includes a reduction in the expression of one or more of: IL-4, IL-5, and IL-13. For example, ILC subsets can produce canonical type 2 cytokines IL-5, IL-9 and IL-13 in response to IL-25 and IL-33, including type 2 innate lymphoid cells (ILC2 cells). In various embodiments, ILC2 cells are responsive to to helminths and allergens, such as Alternaria alternate or papain. In various embodiments, the lung and/or airway tissue includes bronchiolar and/or aveolar tissue. In various embodiments, the lung and/or airway tissue includes epithelial tissue. In various embodiments, treating inflammation includes a reduction in the number of innate lymphoid cells (ILCs). Generally, it is understood that type 1 ILCs include cells that can produce type 1 cytokines (notably IFNγ and TNF) and include NK cells and ILC1s, type 2 ILCs can produce type 2 cytokines (e.g. IL-4, IL-5, IL-9, IL-13), are capable of secreting type 2 cytokines in response to helminth infection, type 3 ILCs are produce cytokines IL-17A and/or IL-22 and include ILC3s and lymphoid tissue-inducer (LTi) cells. In various embodiments, the ILC are type 2 ILCs (ILC2) cells. In various embodiments, ILC2 require IL-7 for differentiation or activation. In various embodiments, ILC2 cells modulate—RORα, GATA3, and/or STAT5 pathways. In various embodiments, the ILC2 cells are capable of promoting the differentiation of naive CD4+ T cells into Th2 cells. In various embodiments, the ILC2 cells do not express one or more of: CD3, CD14, CD16, CD19, CD20, CD56, CD235a, CD1a, and CD123. In various embodiments, the ILC2 cells express one or more of: CD45, CRTH2, CD127 and CD161. In various embodiments, ILC2s can include cells that do not expression one or more of CD3, CD45R, Gr-1, CD11c, CD11b, Ter-119, NK1.1 and TCR-γδ, and can further include cells expressing one or more of CD45, CD90, IL-2Rα, IL-33R and IL-7Rα. In various embodiments, treating inflammation includes a reduction in STAT5 pathway activation in ILCs.

Further described herein is a pharmaceutical composition, including a quantity of a therapeutic agent includes a composition capable of binding or modulating expression of ICOS, ICOS ligand, or both, and a pharmaceutically acceptable carrier. In various embodiments, the composition is an antibody capable of binding to ICOS, ICOS ligand, or both, and a pharmaceutically acceptable carrier. In various embodiments, the composition is capable of modulating ICOS expression and a pharmaceutically acceptable carrier. In various embodiments, the composition is capable of modulating ICOS ligand expression and a pharmaceutically acceptable carrier. In various embodiments, the composition is capable of modulating a type 2 inflammatory response, and a pharmaceutically acceptable carrier. In various embodiments, modulating a type 2 inflammatory response, includes a reduction in the expression of IL-33 and/or IL-25. In various embodiments, modulating a type 2 inflammatory response, includes a reduction in the expression of one or more of: IL-4, IL-5, and IL-13. In various embodiments, the lung and/or airway tissue includes bronchiolar and/or aveolar tissue. In various embodiments, the lung and/or airway tissue includes epithelial tissue. In various embodiments, treating inflammation includes a reduction in the number of innate lymphoid cells (ILCs). In various embodiments, the ILC are type 2 ILCs (ILC2) cells. In various embodiments, the ILC2 cells do not express one or more of: CD3, CD14, CD16, CD19, CD20, CD56, CD235a, CD1a, and CD123. In various embodiments, the ILC2 cells express one or more of: CD45, CRTH2, CD127 and CD161 In various embodiments, treating inflammation includes a reduction in STAT5 pathway activation in ILCs.

Further described herein are methods of modulating inflammation, including selecting a subject in need of treatment for inflammatory related disease and/or condition; and administering a therapeutic agent to the subject, wherein the administration of the composition modulates inflammation in the subject. In various embodiments, the inflammatory related disease and/or condition is acute. In various embodiments, the inflammatory related disease and/or condition is chronic. In various embodiments, the inflammatory related disease and/or condition is a lung related disease and/or condition. In various embodiments, modulating inflammation includes modulating a type 2 inflammatory response, and a pharmaceutically acceptable carrier. In various embodiments, modulating inflammation includes modulating a type 2 inflammatory response, includes a reduction in the expression of IL-33 and/or IL-25. In various embodiments, modulating inflammation includes modulating a type 2 inflammatory response, includes a reduction in the expression of one or more of: IL-4, IL-5, and IL-13. In various embodiments, the lung and/or airway tissue includes bronchiolar and/or aveolar tissue. In various embodiments, modulating inflammation in the subject includes decreased type 2 ILCs (ILC2) cell phenotype. In various embodiments, the ILC2 cells do not express one or more of: CD3, CD14, CD16, CD19, CD20, CD56, CD235a, CD1a, and CD123. In various embodiments, the ILC2 cells express one or more of: CD45, CRTH2, CD 127 and CD161

Described herein are methods and compositions for treatment of inflammation, such as inflammation in lung and/or airway tissue, including asthma. Based on the increasing knowledge that innate immunity plays a role in asthma disease initiation and progression, innate lymphoid cells (ILCs), such as type 2 ILC2s, are herein described as capable of IL-33 signaling activation, further including expression of ICOS, and leading to the induction of airway hyperresponsiveness (AHR) and inflammation in the lungs. Further described is the hereto unknown discovery that ICOS-ligand is expressed in ILC2s, and that ICOS binding of ICOS to ICOS-ligand is required for its function in ILC2s. Using humanized mouse model, adoptive transfer shows that ICOS:ICOS-Ligand interaction is required for efficient function of human ILC2s in vivo. It is further shown that human and mouse ILC2s express ICOS and ICOS-Ligand and that ICOS:ICOS-Ligand interaction provides a survival signal for ILC2s. More specifically, the frequency of dead cells is increased in the absence of ICOS while the frequency of early and late apoptotic ILC2s are comparable suggesting that the rate of proliferation of ILC2s is similar in ICOS deficient and WT mice and that while ICOS provides a survival signal for ILC2s, it appears to be redundant for ILC2s' proliferation.

ICOS:ICOS-Ligand interaction further provides for efficient cytokine production by ILC2s. STAT5 signaling pathway is impaired in ICOS−/− ILC2s, while it is enhanced through ICOS signaling leading to higher cytokine production, thereby providing a mechanism by which survival and cytokine production diminishes in the absence of ICOS or blockade of ICOS:ICOS-Ligand signaling.

While IL-33 treatment induces AHR in control mice, such treatment cannot induce AHR in mice receiving treatment anti-ICOS-ligand antibodies. Blocking ICOS:ICOS-Ligand interaction impairs STAT5 signaling and IL-13 production in ILC2s. As ILC2s are the only cells that express ICOS and ICOS-Ligand, the Inventors' findings set the stage for designing new therapeutic approaches for asthma where ILC2s can be targeted, for instance by dual specific antibodies that recognize ICOS and ICOS-Ligand.

### EXAMPLE 1

### Mice and In Vivo Experiments

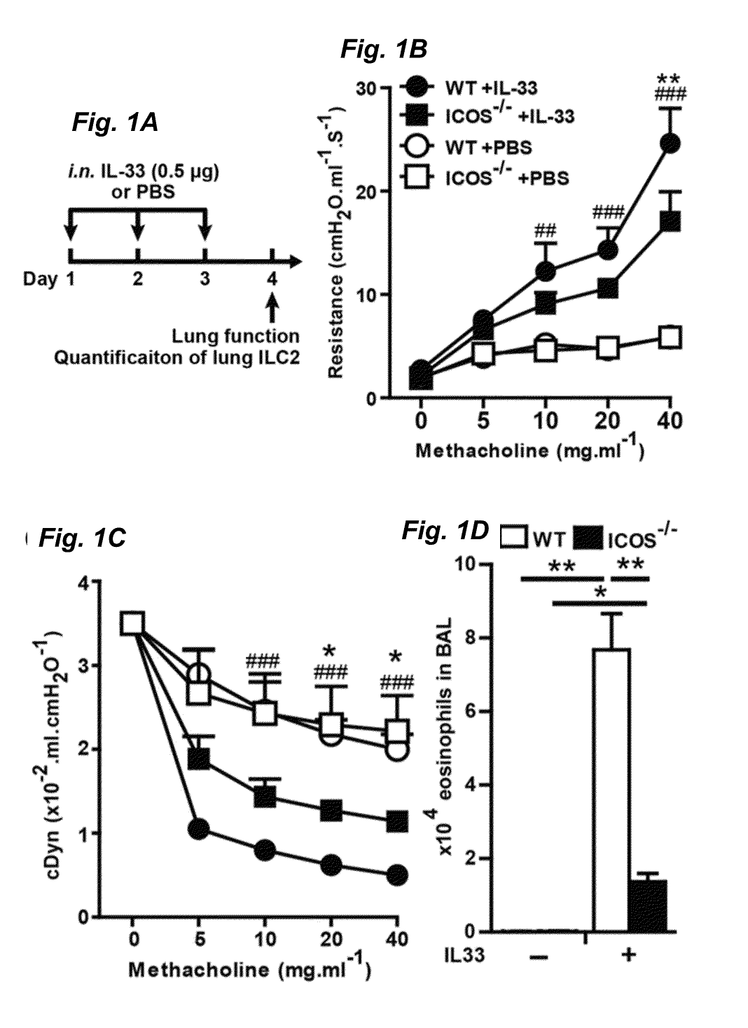

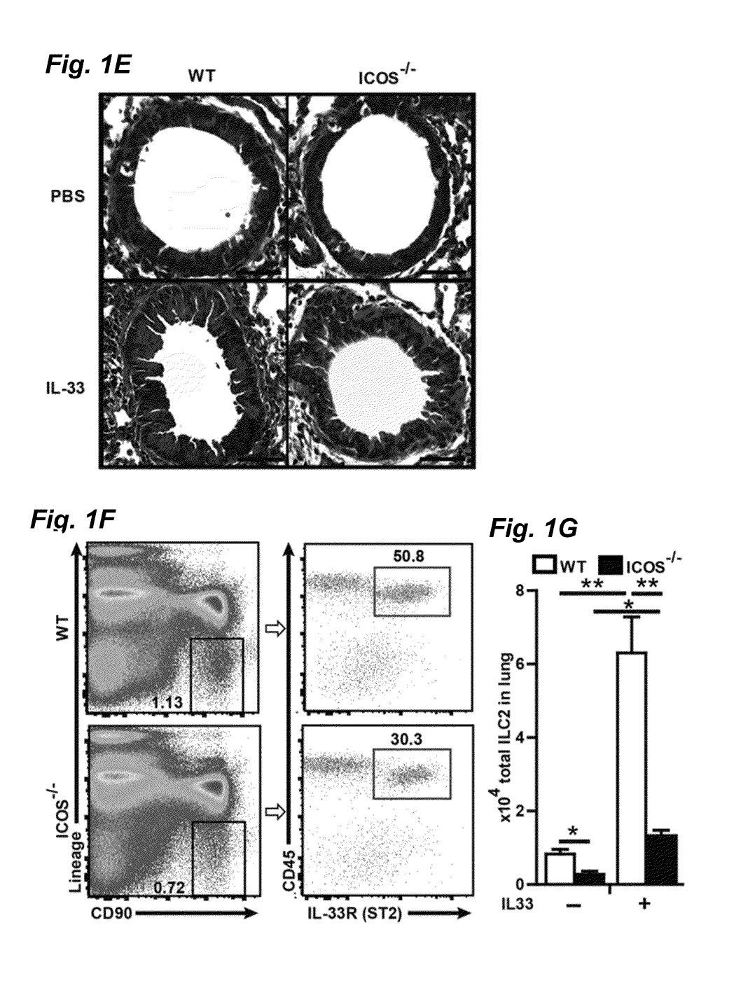

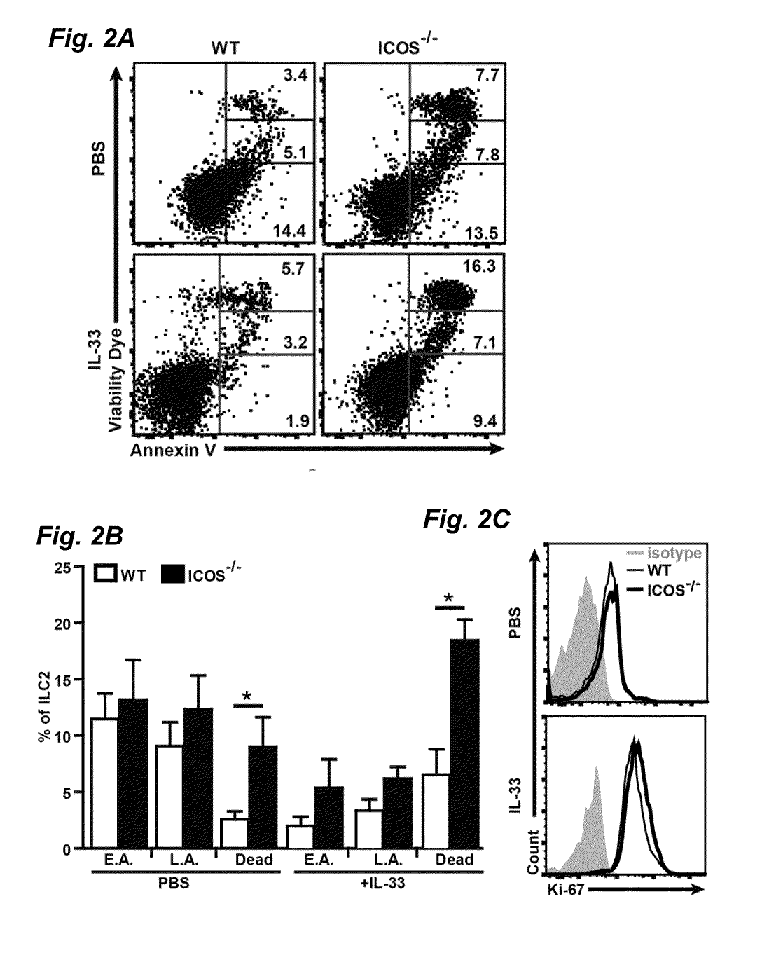

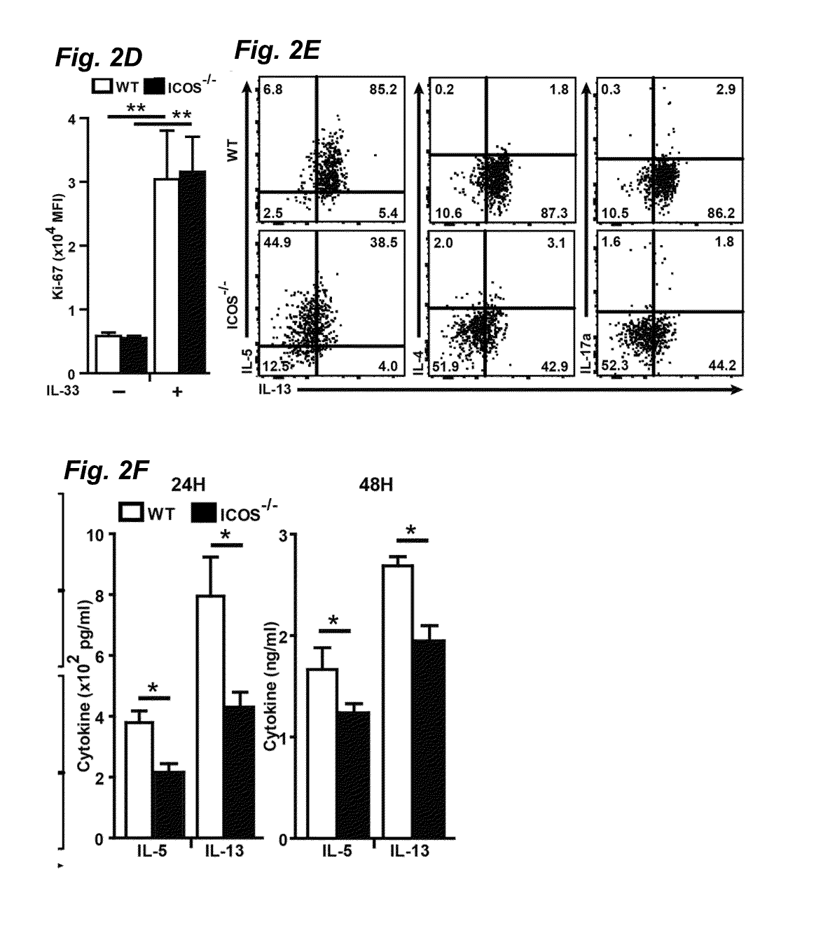

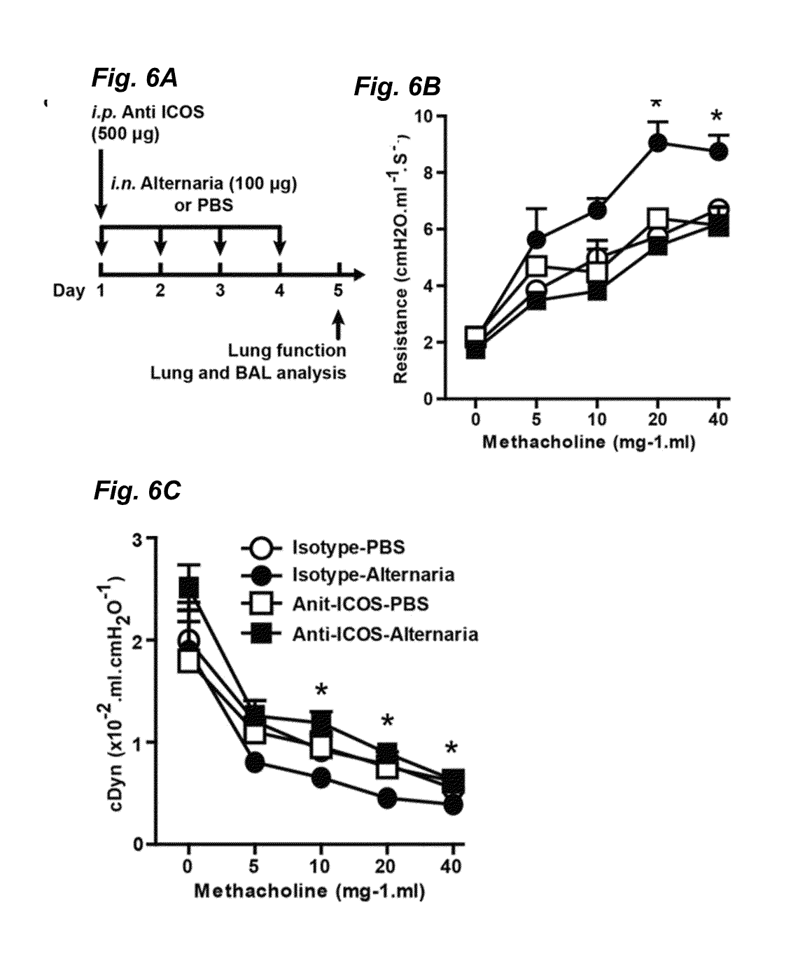

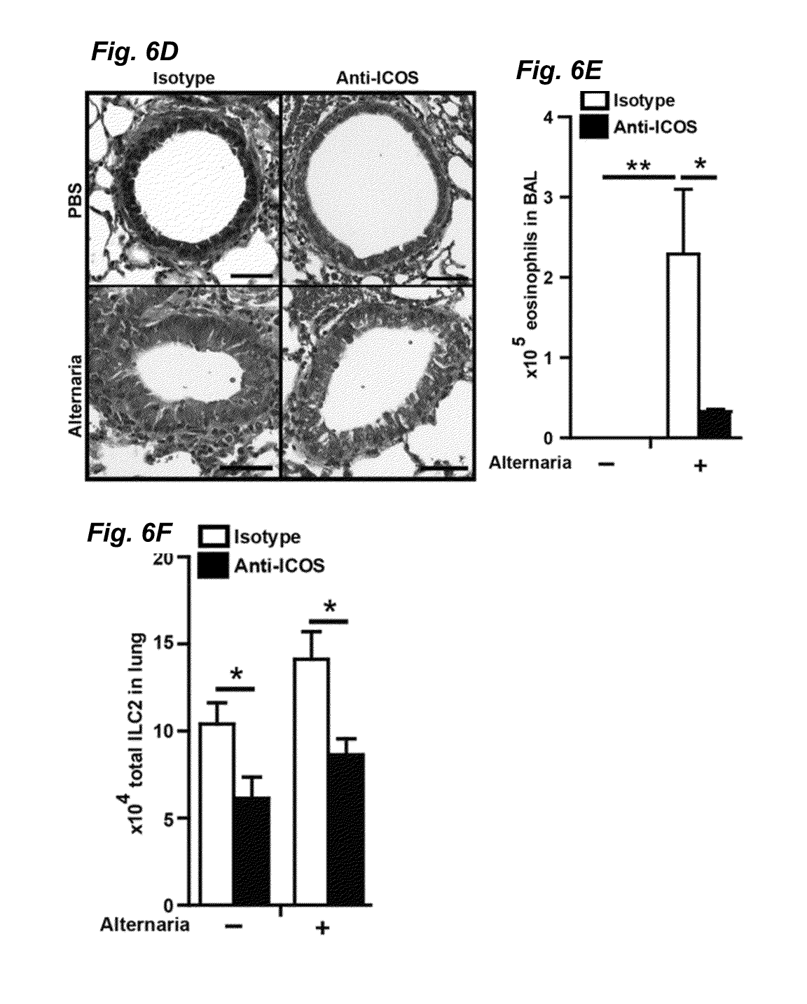

ICOS deficient mice were obtained and backcrossed 11 times to BALB/cByJ as previously described. RAG2 deficient (C.B6(Cg)-Rag2^{tm1.1Cgn}/J), RAG2 GC deficient (C;129S4-Rag2^{tm1.1Flv}Il2rg^{tm1.1Flv}/J) breeder pairs and BALB/cBYJ experimental mice were purchased from the Jackson Laboratory (Bar Harbor, Me.). RAG2 deficient, RAG2 GC deficient and ICOS deficient mice were bred in the Inventors' animal facility at USC. 5-8 weeks age-matched female mice were used in the studies. For in vivo stimulation studies described in FIGS. 1, 4 and 5, carrier free recombinant mouse IL-33 (Biolegend, San Diego, Calif., 0.5 μs/mouse in 50 μl) or PBS (50 μl) was administered intranasally to mice on three consecutive days. One day after the last intranasal stimulation lung function was measured, mice were euthanized and samples were taken. For Alternaria experiments described in FIG. 6, Alternaria alternata (Greerlabs, Lenoir, N.C., 100 μs/mouse in 50 μl) or PBS (50 μl) was administered intranasally on four consecutive days followed by measurement of lung function and sample withdrawal one day after the last intranasal challenge. For in vivo inhibition of ICOS:ICOSL interaction mice received blocking anti-mouse ICOS (Clone: 17G9, 250 μg/ml , BioxCell, West Lebanon, N.H.) or Rat IgG2b (Clone: LTF-2, 250 μg/ml, BioxCell, West Lebanon, N.H.) intraperitoneally. For experiments reported in FIG. 2, mouse ILC2 cells were purified from the lung of either BALB/cBYJ or ICOS deficient mice based on the lack of expression of classical lineage markers (CD3e, CD45R, Gr-1, CD11c, CD11b, Ter119, NK1.1, TCR-γδ and FCεRI) and expression of CD45, ST2, and CD117, using BD FACS ARIA III cell sorter with >95% purity then cells injected to RAG2 GC double knockout mice intravenously followed by intranasal administration of IL-33 as described above.

### EXAMPLE 2

### Flow Cytometry Antibodies and Reagents

Biotinylated anti-mouse lineage (CD3e, CD45R, Gr-1, CD11c, CD11b, Ter119, NK1.1, TCR-γδ and FCεRI), Streptavidin-FITC, Streptavidin-BV510, BV421 anti-mouse CD25, BV510 anti-mouse CD90.2, PE Annexin V, Annexin V binding buffer were purchased from Biolegend (San Diego, Calif.). APC anti-mouse CD127, PerCP-eFluor® 710 anti-Mouse ST2 (IL-33R), Streptavidin APC-eFluor® 780, PE anti-mouse ICOS (CD275), PE/Cy7 anti-mouse CD117 (c-kit), FITC anti-mouse Sca-1, PE/Cy7 anti-mouse CD45, FITC anti-mouse CD45, PE anti-mouse IL-5, PE/Cy7 anti-mouse IL-13, PE/Cy7 anti-mouse IL-4, APC anti-mouse IL-13, eFluor® 660 anti-mouse Ki-67, PE/Cy7 anti-mouse IL-17a, PE anti-mouse pSTAT5 (Y694), PerCP/AF710 anti-mouse pSTAT6 (Y641), Fixation Permeabilization buffer set and Fixable Viability Dye eFluor® 780 were purchased from eBioscience (San Diego, Calif.). BV421 anti-mouse GATA3, BD Cytofix™ Fixation Buffer and BD Phosflow™ Perm Buffer III were purchased from BD biosciences (San Jose, Calif.).

### EXAMPLE 3

### Humanized Mice and Purification of Human ILC2

For human peripheral ILC2, peripheral blood mononuclear cells (PBMCs) were first isolated from human fresh blood by diluting the blood 1:1 in PBS then adding to SepMate™-50 separation tubes (STEMCELL Technologies Inc, Vancuver, Canada) prefilled with 15-ml Lymphoprep™ each (Axis-Shield, Oslo, Norway) and centrifugation at 1200×g for 15 minutes. Human PBMCs were then stained with antibodies against human lineage markers (CD3, CD14, CD16, CD19, CD20, CD56, CD235a, CD1a, CD123), CRTH2, CD161, CD127 and CD45. Thereafter, ILC2s were defined as CD45⁺ lineage− CRTH2⁺ CD127⁺ CD161⁺ and purified by flow cytometry and using BD FACS ARIA III (BD biosciences, San Jose, Calif.) with the purity of >95% (supplementary FIG. 2). Purified human ILC2s were cultured with rh-IL2 (20 ng/ml) and rh-IL-7 (20 ng/ml) for 48 hours then adoptively transferred to RAG2 Il2rg double knockout mice (2×10⁴ cells/mouse) followed by i.n. administration of recombinant human IL-33 (0.5 μs/mouse) or PBS i.n. on day 1-3. On day 1, both groups received either anti-human (clone: 9F.8A4, 500 μs/mouse)+anti-mouse ICOS-ligand (clone: 16F.7E5, 500 μs/mouse) or isotype-matched control (500 μs/mouse). On day 4 lung function was measured and BAL was performed and analyzed. Anti-ICOS-Ligand antibodies were generated as previously described. See Akbari, O., et al. Antigen-specific regulatory T cells develop via the ICOS-ICOS-ligand pathway and inhibit allergen-induced airway hyperreactivity. Nat Med. 2002; 8(9):1024-32, which is fully incorporated herein by reference.

### EXAMPLE 4

### Cytokine Measurement in the Supernatant

Human IL-5 ELISA MAX™ Deluxe was purchased from Biolegend, Ready-SET-Go! ® ELISA for human IL-13, mouse IL-5 and IL-13 were purchased from eBioscience and the level of cytokines were measured according to the manufacturer's instructions.

### EXAMPLE 5

### Measurement of Lung Function

Lung function was evaluated by direct measurement of lung resistance and dynamic compliance in restrained tracheostomized mechanically ventilated mice using FinePointe RC system (Buxco Research Systems, Wilmington, N.C.) under general anesthesia as described before. In brief, mice were anesthetized using Ketamin (100 mg/Kg body weight) mixed with Xylazine (10 mg/Kg) then tracheostomized and attached to FinePointe RC system with ventilation rate of 140 breath/minutes. Lung resistance and dynamic compliance were measured in 3 minutes period after exposing to increasing doses of aerosolized methacholine.

### EXAMPLE 6

### Collection of Bronchoalveolar Lavage (BAL) Fluid and Lung Histology

Mice were euthanized after evaluating lung function trachea was intubated and lungs were washed three times with 1 ml of PBS then the cells were harvest by centrifugation at 400×g for 7 minutes as previously described. Relative and absolute cell number in the BAL were counted using flow cytometry. In brief, cells were stained with PE-anti-SiglecF (BD biosciences), FITC-anti-CD19, PerCP/Cy5.5-anti-CD3e, APC-anti-Gr-1, PE/Cy7-anti-CD45, APC/Cy7-anti-CD11c (All from Biolegend) and eFluor450-anti-CD11b (eBioscience) in the presence of anti-mouse FC-block (BioXcell, West Lebanon, N.H.). Thereafter cells were washed twice with PBS+1% BSA, and after adding countBright absolute count beads (Life Technologies, Grand Island, N.Y.) at least of 1×10⁴ CD45⁺ cells were acquired on BD FACSCANTO-II (BD biosciences). Data were analyzed using the latest version of FlowJo (Treestar, Ashland, Oreg.).

After the BAL was performed, transcardial perfusion of lungs was performed with PBS and subsequently lungs were fixed and harvested for histology in 4% paraformaldehyde buffered in PBS. After fixation, the lungs were embedded in paraffin, cut into 4 μm sections and stained with H&E. Histology pictures were acquired using Keyence BZ-9000 microscope (Keyence, Itasca, Ill.) and analyzed using BZ-II Image Analysis Application (Keyence, Itasca, Ill.).

### EXAMPLE 7

### Statistical Analysis

Experiments were repeated at least three times (N=4-6 each) and data are shown as the representative of 3 independent experiments. AHR data were analyzed by repeated measurements of general linear model. Al the other data were analyzed using JMP statistical software (Cary, N.C.) by Student's t-Tests and confirmed by Mann-Whitney U.

### EXAMPLE 8

### ICOS Deficient Mice Show Reduced IL-33 Induced AHR, Inflammation and Pulmonary ILC2s

It has been reported, that murine ILC2s express ICOS and can be activated through IL-33 signaling leading to the induction of AHR and inflammation in the lungs. The Inventors first addressed the question whether the expression of ICOS is required for the function of ILC2s by assessing the level of IL-33 induced AHR and airway inflammation. To this aim the Inventors used ICOS deficient mice on BALB/C background, activated pulmonary ILC2 by intranasal administration of IL-33 and compared the induction of AHR and inflammation in ICOS deficient with that of wild type BALB/c mice. As shown in FIG. 1A, mice received i.n. of either IL-33 (0.5 μg in 50 μl per mouse) or PBS (50 μl per mouse) on three consecutive days. One day after the last i.n. challenge, lung function was measured by direct measurement of lung resistance and dynamic compliance in anesthetized tracheostomized mice using FinePointe RC system (Buxco Research Systems), as described in methods, followed by analyzing bronchial alveolar lavage (BAL) and taking lung tissue samples. As expected, i.n. administration of IL-33 significantly increased lung resistances compared to PBS in wild type mice (P<0.005 at dose 40, FIG. 1B). Interestingly, lung resistance in IL-33-treated ICOS^{-/-} mice was significantly lower compare to that of IL-33-treated WT mice (P<0.01 at dose 40, FIG. 1B), however, it was higher than lung resistance in PBS-treated ICOS^{-/-} mice (P<0.01 at dose 40, FIG. 1B) indicating that IL-33-induced AHR is lower in ICOS^{-/-} mice. In agreement with lung resistance, results of dynamic compliance showed improved response in IL-33 treated ICOS^{-/-} compared to IL-33 treated WT mice while they showed significantly lower dynamic compliance compare to their PBS-treated counterparts (FIG. 1C). Analyzing the contents of bronchoalveolar lavage (BAL) showed that the number and the frequency of eosinophils are dramatically reduced in IL-33 treated ICOS^{-/-} compared to WT mice indicating that IL-33 induced inflammation is impaired in the absence of ICOS (P<0.01 absolute number and P<0.05 frequency, FIG. 1D-E). IL-33 treatment results in increased frequency and absolute number of eosinophils as compared to PBS treatment in both WT and ICOS^{-/-} mice (P<0.01, FIG. 1D-E).

As shown in FIG. 1F-G, IL-33 treatment significantly increased the total number and the frequency of pulmonary ILC2s in WT and in ICOS^{-/-} mice (P<0.01). Interestingly, the Inventors found that the number and the frequency of pulmonary ILC2s is dramatically lower in ICOS^{-/-} mice compare to WT controls in PBS and in IL-33 treated groups (P<0.05 in PBS, P<0.01 in IL-33 treated mice, FIG. 1F-H). These results suggest that ICOS is playing an important role for the function and homeostasis of pulmonary ILC2s.

### EXAMPLE 9

### Lack of ICOS Increases Cell Death and Reduced STAT5 Signaling

Since the number of ILC2 are lower in the Inventors next addressed the question whether lack of ICOS affects the survival or proliferation of ILC2s. A cohort of ICOS^{-/-} and WT mice were intranasally challenged with rm-IL-33 (0.5 μs/mouse) and after 24 hours pulmonary ILC2s were stained with dead cell discrimination dye, Annexin V for analyzing cell death and apoptosis. Expression of Ki-67 was analyzed as an indicator of proliferation. The Inventors' data shows that the number of dead cells is significantly increased in PBS-treated ICOS^{-/-} mice compare to PBS-treated WT mice (P<0.05, FIG. 2A-B). Similarly, the number of dead ILC2s in IL-33-treated ICOS^{-/-} mice is dramatically increased as compared to IL-33-treated WT mice (P<0.05, FIG. 2A-B) whereas, the number of early apoptotic and late apoptotic ILC2s are comparable in both strains and treatments. Moreover, the Inventors' data reveal that there is no significant difference between the expression level of Ki-67 in ICOS^{-/-} and WT ILC2s in PBS or IL-33 treated mice (FIG. 2C-D). These data suggest that lack of ICOS impairs the survival of pulmonary ILC2s rather than their proliferation.

The Inventors next examined whether ICOS is required for functional production of cytokines by ILC2s by intracellular measurement of cytokines 24 hours after intranasal administration of rm-IL33 and by measurement of cytokines in the supernatant of in vitro culture of purified pulmonary ILC2s for 24 and 48 H. For intracellular staining fixable dead cell discrimination dye was used to assess the level of cytokine production specifically in live ILC2s. Intracellular cytokine staining data show that production of IL-13 by ILC2s are dramatically lower in ICOS^{-/-} compare to WT mice, whereas there is no difference in the production of IL-5 between ICOS^{-/-} and WT mice (FIG. 2E). The Inventors did not detect a significant production of IL-4 or IL-17a by ILC2s. Interestingly, the Inventors observed a significantly lower level of IL-5 and IL-13 in the supernatant of in vitro cultured purified ICOS^{-/-} ILC2s as compared to WT ILC2s at 24 and 48 hours after culture (*<P<0.05, FIG. 2F). Taken together, these data suggest that while production of IL-13 is affected by lack of ICOS, IL-5 production in ICOS^{-/-} is also ultimately lower due to lower number of viable ILC2s in these mice.

### EXAMPLE 10

### Lack of ICOS Impairs STAT5 Signaling in Pulmonary ILC2s

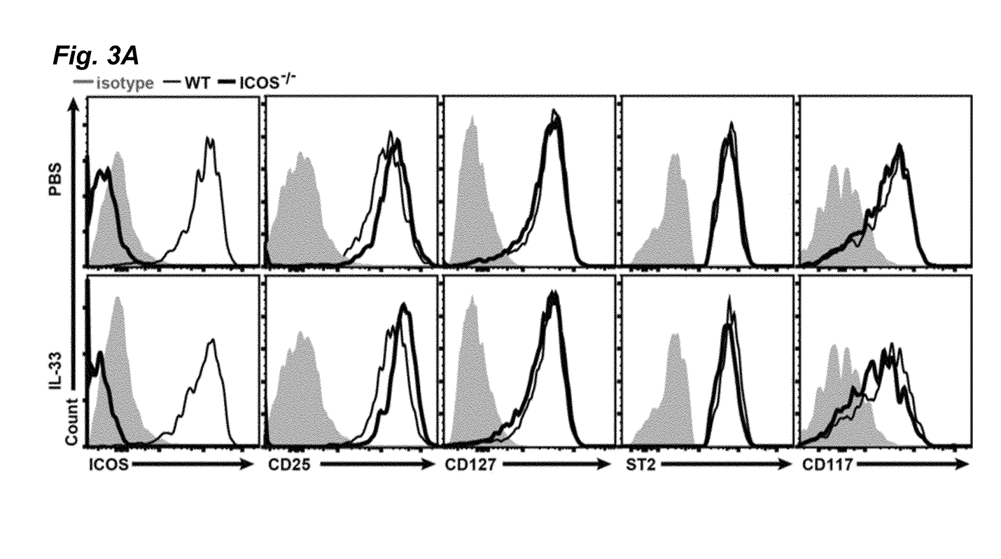

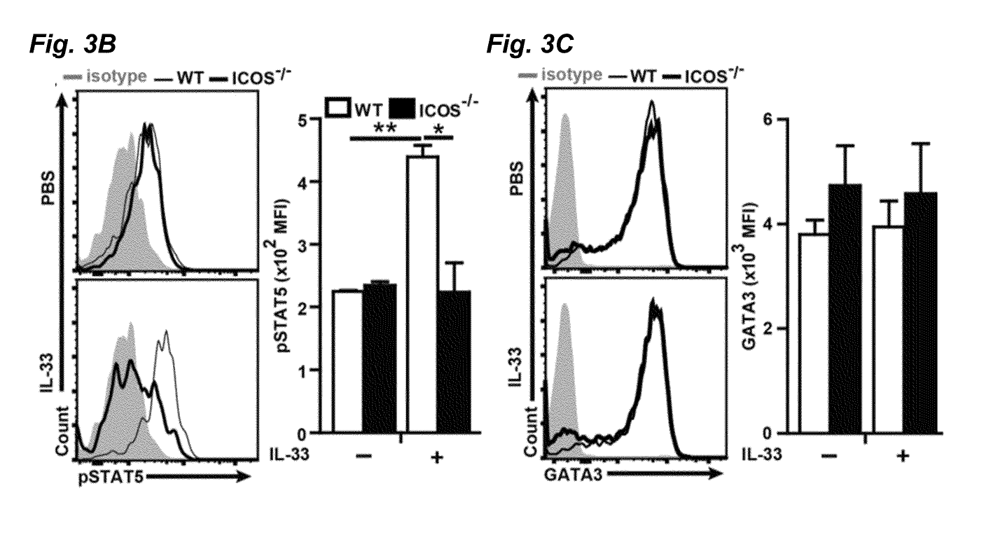

Since the Inventors found that survival of ILC2s is reduced in the absence of ICOS the Inventors aimed to investigate whether the expression of the receptors that might mediated the survival of ILC2s are altered in ICOS^{-/-} mice. Therefore, the Inventors evaluated the expression of CD25, CD127, ST2 and CD117 in pulmonary ILC2s in ICOS^{-/-} and compared it to those of WT mice in steady state (PBS treated) and after stimulation with IL-33. To confirm the phenotype of ICOS^{-/-} mice, the level of ICOS was also evaluated in both strains. The Inventors' results show that while there is no significant difference between the level of CD127, ST2 and CD117, the level of CD25 is increased in ICOS^{-/-} mice suggesting an altered sensitivity to IL-2 in the absence of ICOS (FIG. 3A).

Since the Inventors observed that the level of IL-2Rα is increased in ICOS^{-/-} mice to examine the altered sensitivity to IL-2 the Inventors tested the level of phosphorylation of Signal Transducer and Activator of Transcription 5 (STAT5) in response to IL-2 stimulation in ILC2s. To reach this goal, ICOS^{-/-} and WT mice were challenged i.n. by either rm-IL-33 (0.5 μg/mouse) or PBS. After 24 h lungs single cells were stimulated with recombinant murine IL2 (100 ng/ml) for 30 minutes then ILC2s were analyzed for the expression of phosphoSTAT5. The Inventors' results show that although in PBS treated WT and ICOS^{-/-} mice the level of phosphoSTAT5 in ILC2s seems to be comparable, IL-33 treated ICOS^{-/-} ILC2s show significantly lower level of phosphoSTAT5 compare to that of WT mice (FIG. 3C). These results suggest a reduced sensitivity to IL-2 signaling despite higher expression of IL-2Rα in ICOS^{-/-} pulmonary ILC2s.

To further investigate the mechanism of impaired cytokine production in ILC2s the Inventors evaluated the level of transcription factor GATA binding protein-3 (GATA-3) that have been associated with development and maintenance of ILC2s. ICOS^{-/-} or WT mice received i.n. administration of either rm-IL-33 (0.5 μg/mouse) or PBS followed by analyzing the level of GATA-3 in pulmonary ILC2s 24 hours later. The Inventors' results show that there is no difference in the level of GATA-3 in ILC2s between ICOS^{-/-} and WT in either IL-33 or PBS treatments. These results suggest that GATA-3 pathway in ILC2s is not affected by lack of ICOS.

### EXAMPLE 11

### Adoptively Transferred ICOS^{-/-} ILC2s Fail to Induce AHR in RAG2^{-/-} Il2rg^{-/-} Hosts

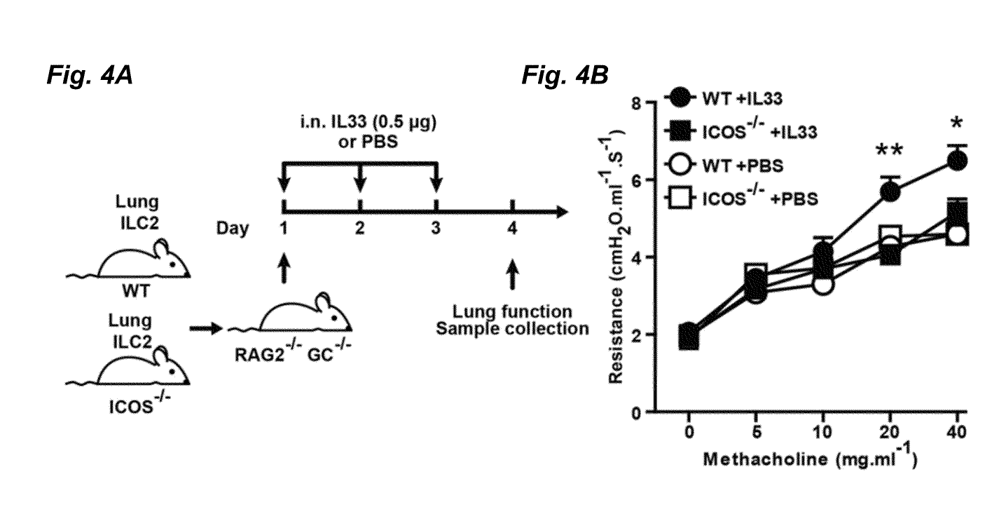

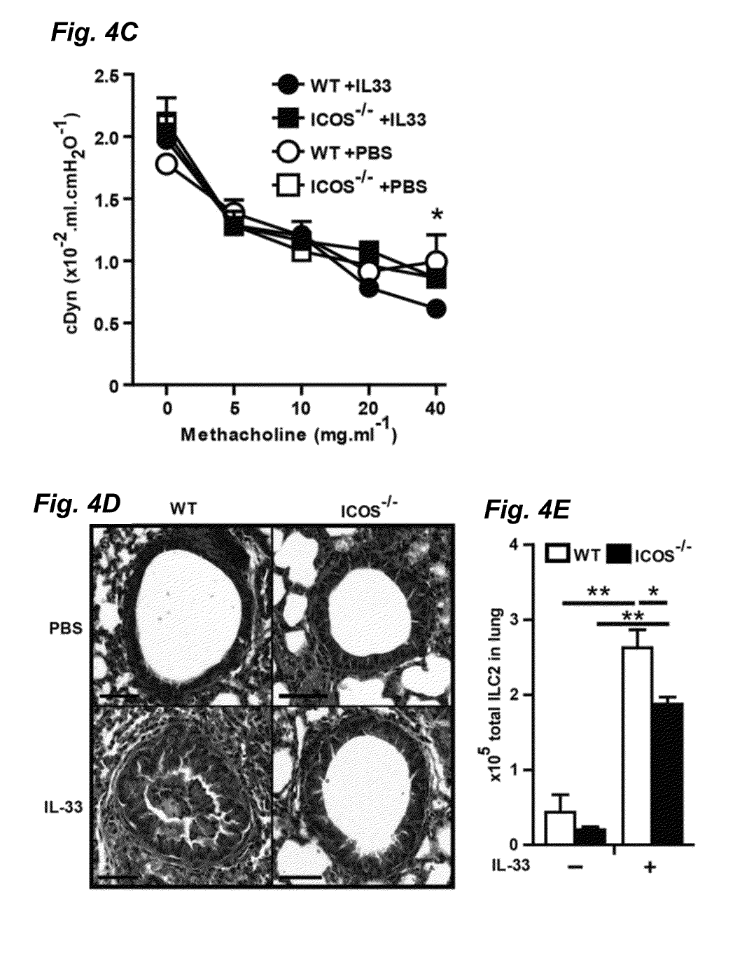

The Inventors found that IL-33-induced AHR and airway inflammation is mice that lack ICOS in all their cells. To confirm these findings and to eliminate the effect of bystander cells including T cells subsets which can express IL-33 receptor, a series of adoptive transfer experiments were performed. As shown in FIG. 4A, pulmonary ILC2s from a cohort of WT and ICOS^{-/-} mice were purified using flow cytometry as mentioned in detail in the methods section and 1.5×10⁴ purified ILC2s from either strain were injected into RAG2 Il2rg double knockout mice on day 1 followed by i.n. administration of either IL-33 (0.5 μg/mouse) or PBS on day 1-3 and evaluation of lung function and inflammation on day 4. The Inventors' data show that compared to PBS, i.n. administration of IL-33 significantly increased lung resistance and dynamic compliance in the recipients of WT ILC2s (P<0.05 at dose 40, FIG. 4B-C). Interestingly, IL-33 treated recipient of ICOS^{-/-} ILC2s did not show a significant increase compare to PBS treated recipients of ICOS^{-/-} ILC2s (FIG. 4B-C). In fact IL-33 treated recipient of WT ILC2s showed significantly higher lung resistance and lower dynamic compliance compared to those of IL-33 treated recipient of ICOS^{-/-} ILC2s (P<0.05 at dose 40, FIG. 4B-C). Lung histology shows that while IL-33 treatment caused recruitment of inflammatory cells and thickening of epithelium in the recipient of WT cells, IL-33 treated recipients of ICOS^{-/-} deficient cells show alleviated inflammation (FIG. 4D). Analyzing number of ILC2s in the lungs revealed that IL-33 treatment dramatically increases the number of transferred WT and ICOS^{-/-} ILC2s (P<0.01, FIG. 4E). However, the number of transferred ICOS^{-/-} ILC2s in IL-33 treated mice is significantly lower compare to that of WT ILC2s (P<0.05, FIG. 4E). These data indicate that ICOS deficient ILC2s fail to induce AHR upon IL-33 stimulation.

### EXAMPLE 12

### Blocking ICOS in RAG2^{-/-} Mice Hinders IL-33-Induced AHR and Lung Inflammation

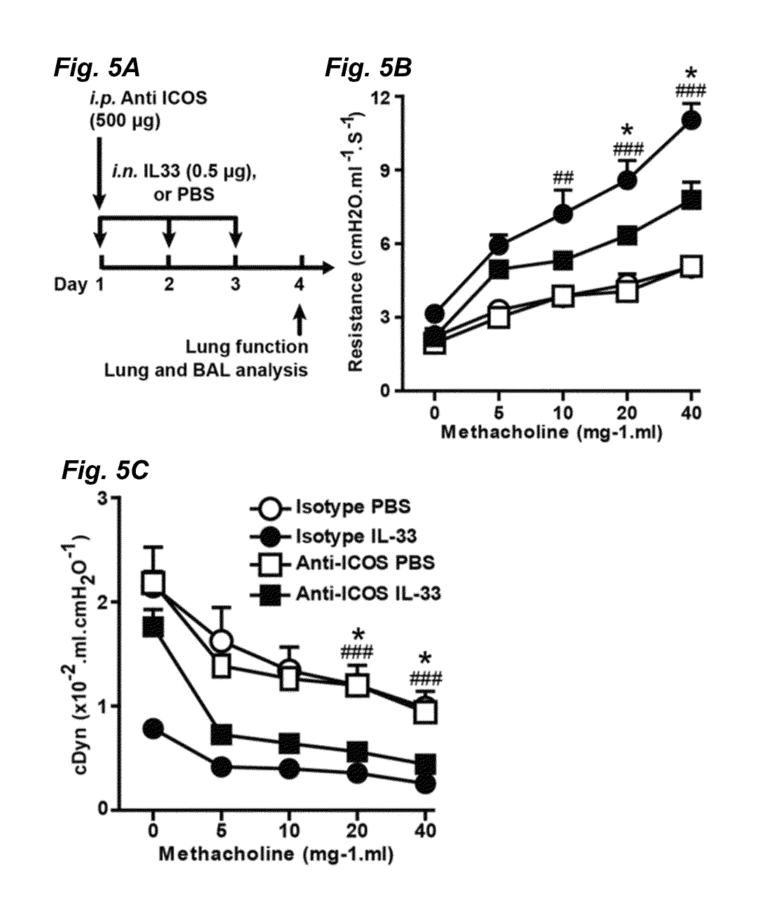

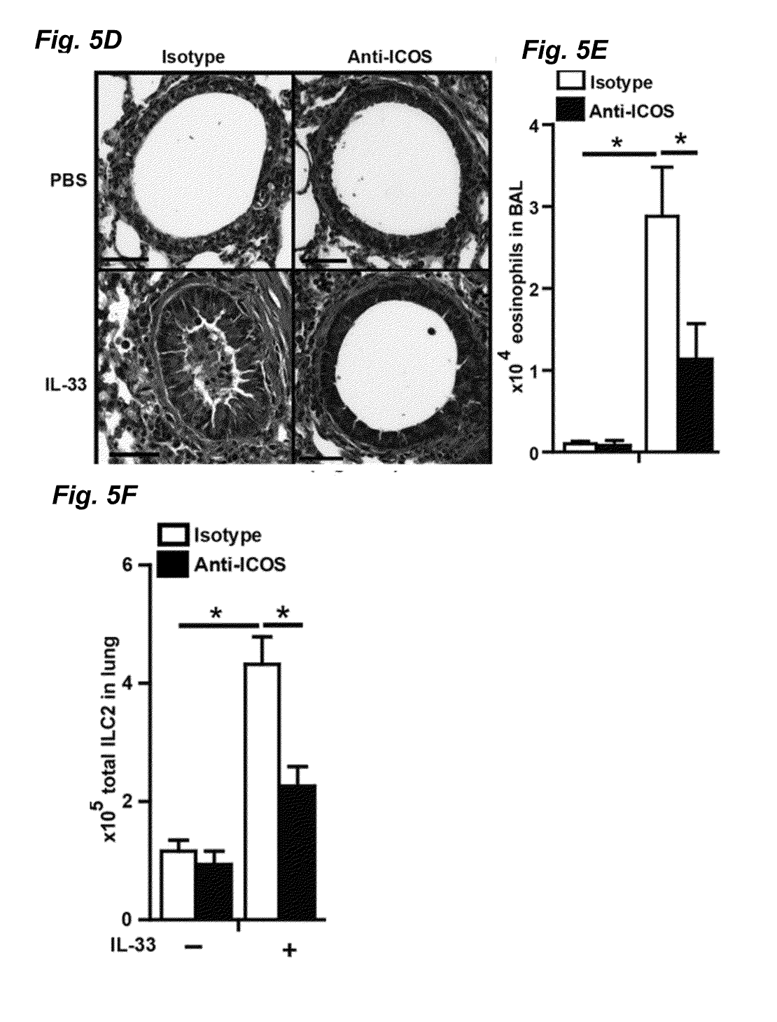

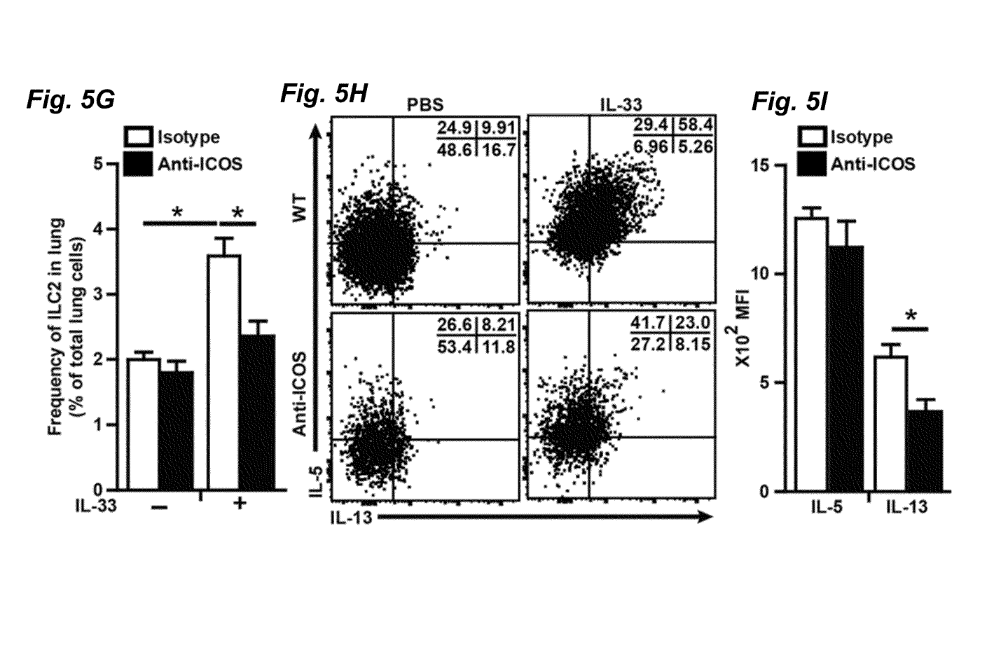

Next the Inventors addressed the question whether blocking ICOS-ICOSLigand binding leads to the same results as the Inventors observed in ICOS^{-/-} mice. Thus the Inventors examined the effects of anti-ICOS blocking antibody on IL-33 induced AHR and lung inflammation in RAG2^{-/-} mice that lack recombination activating gene 2 resulting the absence of mature B and T cells. The Inventors used RAG2^{-/-} mice because only ILC2s express IL-33R in these mice and administration of IL-33 specifically activates ILC2s in these mice. RAG2^{-/-} mice received either anti-ICOS (500 μs/mouse, clone: 17G9) or Rat IgG2b (isotype matched control) intraperitoneally on day 1 and each group received i.n. either IL-33 (0.5 μg/mouse) or PBS on day 1-3 followed by measurement of lung function and sample acquisition on day 4 (FIG. 5A). Lung function data shows that IL-33 i.n. induces a striking increase in lung resistance (P<0.005 at dose 40, FIG. 5B) and a significant decrease in dynamic compliance (P<0.005 at dose 40, FIG. 5C) in WT. Interestingly, lung resistance in IL-33 treated mice that received anti-ICOS was significantly lower and dynamic compliance higher than in IL-33 treated isotype control receiving mice (P<0.05 at dose 40, FIG. 5B-C).

Examining lung histology shows that in isotype control, but not anti-ICOS receiving mice, IL-33 leads to thickening of epithelium and increased inflammatory cells (FIG. 5D). Similarly, the number of eosinophils in bronchoalveolar lavage is significantly higher in IL-33 treated than in PBS treated mice, however, its significantly lower in anti-ICOS treated than isotype treated mice (P<0.05, FIG. 5E). Analyzing the number and frequency of ILC2s in the lung reveals that anti-ICOS antibody significantly reduced the number and frequency of pulmonary ILCs compared to isotype control (P<0.05, FIG. 5F-G). Besides frequency and number the Inventors further analyzed the cytokine production in pulmonary ILC2s by intracellular staining and found that anti-ICOS administration leads to significantly lower production of IL-13 but not IL-5 than isotype control (P<0.05, FIG. 5H-I). These results indicate that ICOS binding of ICOS to ICOS-ligand is required for its function in ILC2s.

### EXAMPLE 13

### ICOS is Required for the Induction of Allergen-Induced AHR and Lung Inflammation

The Inventors next aimed to investigate whether ICOS is required for the induction of AHR and lung inflammation induced by a clinically relevant allergen. To this aim RAG2^{-/-} mice received i.p. either anti-ICOS (500 μs/mouse, clone: 17G9) or Rat IgG2b on day 1 and i.n. extract of Alternaria alternata (100 μs/mouse) on day 1-4 followed by measurement of lung function and sample withdrawal on day 5 (FIG. 6A). As shown in FIG. 6B-C administration of Alternaria induced AHR, as evident by increased lung resistance and decreased dynamic compliance, only in isotype receiving but not anti-ICOS receiving mice (P<0.05 at dose 40). Lung histology shows an increased thickening of epithelium and increased number of inflammatory cells in Alternaria-treated isotype receiving but not anti-ICOS receiving mice (FIG. 6D). Analyzing the contents of BAL shows a significantly increased number of eosinophils in Alternaria treated mice, however, the number of eosinophils is dramatically lower in anti-ICOS receiving than in isotype receiving mice (P<0.01and P<0.05 respectively, FIG. 6E). Total number of ILC2s is significantly lower in anti-ICOS receiving than in isotype receiving mice (FIG. 6F). These results suggest that ICOS plays an important role in the function of ILC2 in response to clinically relevant allergens.

### EXAMPLE 14

### Pulmonary ILC2s Express Functional ICOS-Ligand

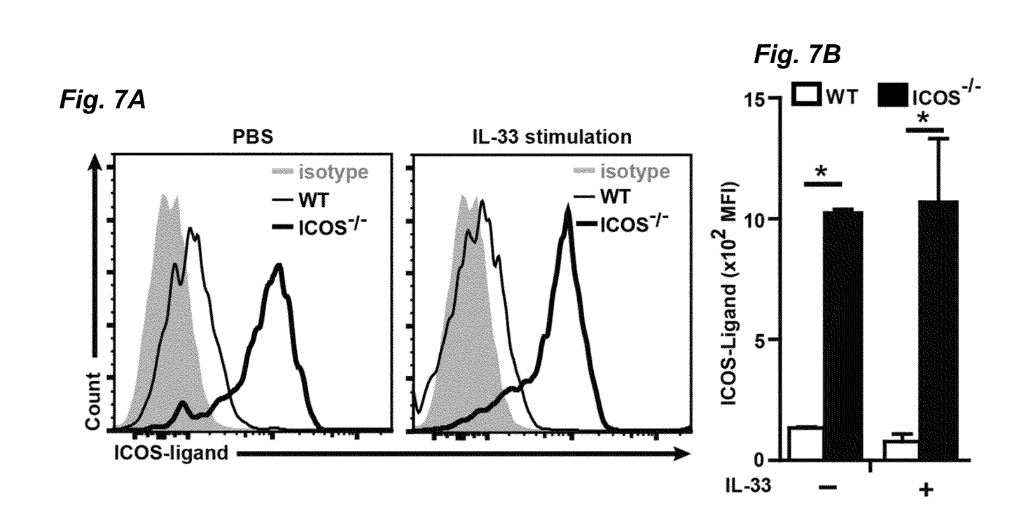

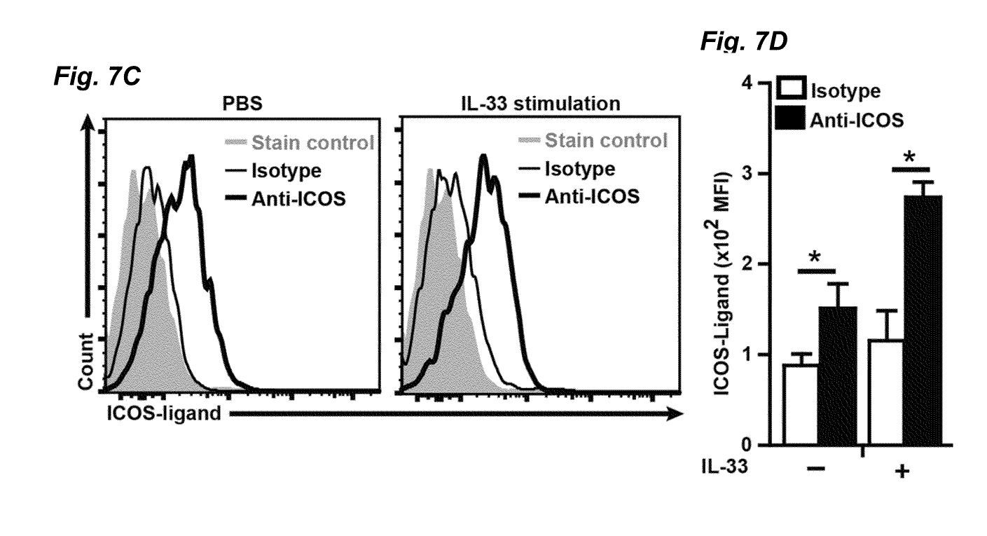

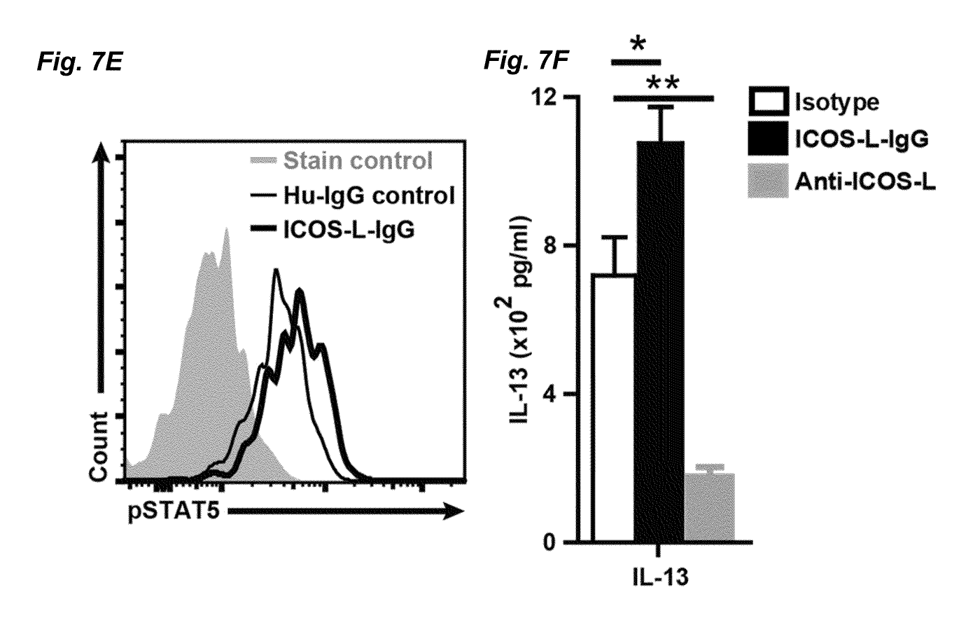

Since the Inventors found that binding ICOS-ICOS-Ligand using antibody shows similar results to ICOS^{-/-} mice and that ICOS is required for cytokine production by purified in vitro cultured ILC2s the Inventors examined whether ILC2s express ICOS-Ligand. To this aim WT and ICOS^{-/-} mice were challenged intranasally by rm-IL33 (0.5 μs/mouse) or PBS and pulmonary ILC2s were analyzed for the expression of ICOS-Ligand by flow cytometry after 24 hours. Surprisingly, the Inventors found, for the first time, that while WT ILC2s express low level of ICOS-Ligand, ICOS ILC2s express strikingly high level of ICOS-Ligand in PBS and IL-33 treated mice (FIG. 7A-B). Since it has been shown that ICOS-Ligand is down-regulated in APCs upon binding to ICOS, the Inventors hypothesized that ICOS-Ligand is down-regulated in WT ILC2s upon binding to ICOS. To test the Inventors' hypothesis the Inventors cultured ILC2s from PBS and IL-33 treated mice in the presence of anti-ICOS (10 μg/ml, clone:17G9) or Rat IgG2b (10 μg/m1) for 24 hours. Results show that while a low level of ICOS-Ligand expression can be detected in cultured ILC2s in the presence of isotype control, the level of expression of ICOS-Ligand is significantly increased in cultured ILC2s in the presence of anti-ICOS antibody (FIG. 7C-D).

To confirm the functionality of ICOS-Ligand in ILC2s the Inventors evaluated the level of phosphoSTAT5 and production of IL-13 by purified cultured pulmonary ILC2s in the presence of plate bound mouse ICOS-Ligand-IgG (5 μg/m1) fusion protein or plate bound human-IgG (isotype control). The culture media was supplemented with rm-IL2 (100 ng/ml), rm-IL-33 (100 ng/ml) and IL-7 (20 ng/ml). The Inventors' results show that the level of phosphoSTAT5 is increased in the presence of ICOS-Ligand-IgG as compared to isotype control (FIG. 7E). Moreover, the Inventors' data show that purified pulmonary ILC2s produce significantly higher level of IL-13 in the presence of ICOS-Ligand-IgG than in the presence of isotype control while IL-13 production is dramatically reduced in the presence of anti-ICOS-Ligand. These data show that pulmonary ILC2s express ICOS-Ligand besides ICOS and that expression of ICOS-Ligand plays a functional role in ILC2s.

### EXAMPLE 15

### Human Peripheral ILC2s Express Functional ICOS and ICOS-Ligand

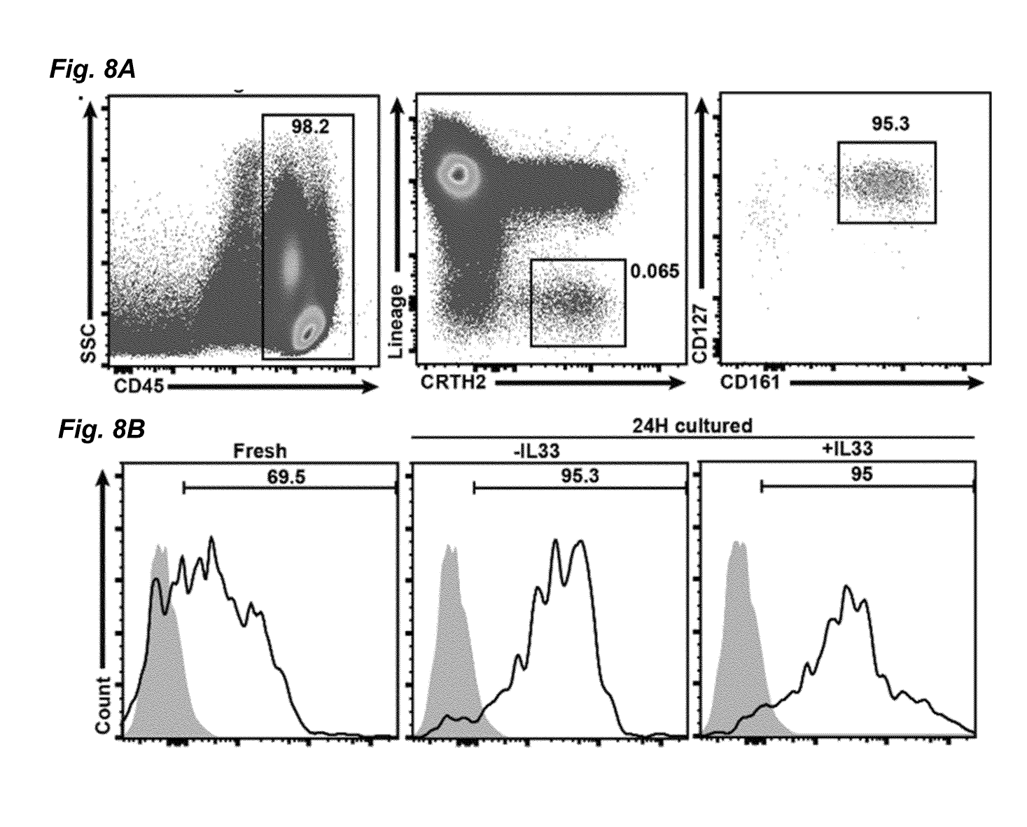

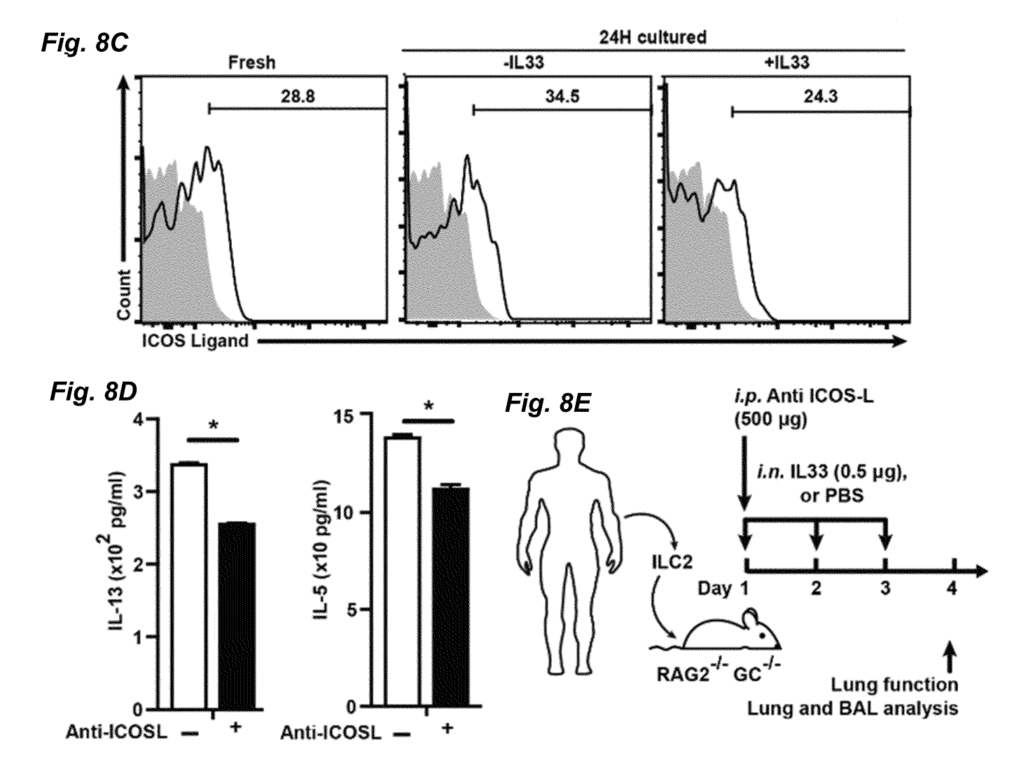

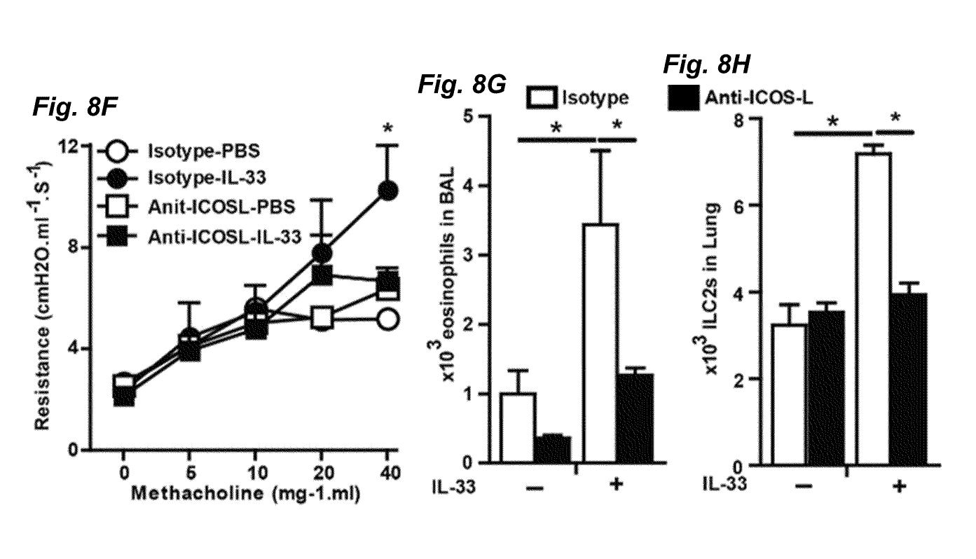

The Inventors next addressed the question whether human ILC2s express ICOS and ICOS-Ligand and whether they play a crucial role in the function of ILC2s. To reach this goal peripheral blood form healthy donors was collected and gated for ILC2s based on the lack of expression of human lineage markers (CD3, CD14, CD16, CD19, CD20, CD56, CD235a, CD1a, CD123), expression of CD45, CRTH2, CD127 and CD161 (FIG. 8A) then analyzed for the expression of ICOS and ICOS-ligand (FIG. 8B-C, left panels). Alternatively, human peripheral blood mononuclear cells (PBMCs) were cultured in the presence of recombinant human (rh)-IL-2 (20 ng/ml), rh-IL-7 (20 ng/ml) in the presence or absence of rh-IL-33 (20 ng/ml) for 24 hours and expression of ICOS and ICOS-Ligand was evaluated by flow cytometry (FIG. 8B-C, right panels). The Inventors found that ICOS is expressed by human peripheral ILC2s at steady state and its expression is increased upon in vitro culture with IL-2 and IL-7, while IL-33 stimulation seems to be redundant for expression of ICOS by human ILC2s (FIG. 8B). Similar to mouse pulmonary ILC2s, the Inventors found that human peripheral ILC2s express ICOS-Ligand a low basal level that is moderately increased after in vitro stimulation by IL-2 and IL-7, while is moderately decreased by IL-33 (FIG. 8C).

To evaluate the functional requirement of ICOS-ICOS-ligand binding for cytokine production by human ILC2s, the Inventors purified ILC2s from PBMCs and cultured in the presence of (rh)-IL-2 (20 ng/ml), rh-IL-7 (20 ng/ml) and rh-IL-33 (20 ng/ml) in the presence of blocking anti-human-ICOS-ligand antibody (10 μg/ml, clone: 9F.8A4) or isotype control for 72 hours followed by measurement of IL-13 and IL-5 in the supernatant by ELISA. The results show that blocking ICOS-ICOS-ligand interaction significantly reduces production of IL-13 and IL-5 by human cultured ILC2s (P<0.05, FIG. 8D).

To further confirm the functional requirement of ICOS-ICOS-Ligand interaction for human ILC2s, the Inventors purified ILC2s from human PBMCs and after 48 h culture in vitro in the presence of (rh)-IL-2 (20 ng/ml) and rh-IL-7 (20 ng/ml) adoptively transferred to RAG2Il2rg double knockout mice, through tail vein, that lack T,B and NK cells and ILCs. Then mice received either anti-human-ICOS-Ligand+anti-mouse-ICOS-Ligand (clone: 9F.8A4 and 16F.7E5, 500 μs/mouse each) or isotype control on day 1 and either i.n. rh-IL-33 (1 μs/mouse) or PBS on days 1-3 (FIG. 8E). On day 4, lung function was measured as described above and assessment of BAL was performed. The results show that IL-33 treatment induces AHR in mice that received isotype control, but failed to induce AHR in mice that received anti-ICOS-ligand antibodies (P<0.05, FIG. 8F). Analyzing the content of BAL shows that IL-33 treatment significantly increases the number and frequency of eosinophils only in isotype control but not anti-ICOS-ligand treated mice (P<0.05, FIG. 8G). The number and frequency of eosinophils are significantly lower in mice that received anti-ICOS-Ligand than in recipient of isotype control (P<0.05, FIG. 8G). Taken together, these results indicate that human peripheral ILC2s express both ICOS and ICOS-Ligand and that ICOS:ICOS-Ligand ineraction plays a crucial role for the function of human ILC2s.

### EXAMPLE 16

### Discussion

In this study the Inventors demonstrate that ICOS is required for ILC-mediated induction of airway hyperreactivity and inflammation in murine models and humanized mice. The Inventors show, for the first time, that mouse and human ILC2s express both ICOS and ICOS-Ligand and that ICOS: ICOS-Ligand interaction is required for efficient ILC2s' function and provides a survival signal for ILC2s. The Inventors demonstrate that blocking ICOS:ICOS-Ligand interaction impairs STAT5 signaling and IL-13 production in ILC2s.

The Inventors found that in the absence of ICOS, IL-33-induced AHR and lung inflammation is reduced. Administration of IL-33 to WT and ICOS^{-/-} mice results in lower lung resistance and higher dynamic compliance in ICOS^{-/-} than in WT mice suggesting that ICOS plays an important role for IL-33-induced ILC2-mediated AHR. Using an IL-33 model to test the functional requirement for ICOS in ILC2s' cytokine production and survival since IL-33 and IL-25 have been previously shown to induce ILC2-mediated AHR and lung inflammation in RAG^{-/-} mice. However IL-33 was reported to be more potent that IL-25 in activating ILC2s. Using IL-33-based system the Inventors found that ICOS^{-/-} mice show reduced lung inflammation, and dramatically lower number of eosinophils in the BAL compare to WT mice. These findings suggest that besides AHR, ILC2s-mediated induction of lung inflammation and eosinophilia depend on ICOS.

In line with the Inventors' findings, it has been shown that ICOS is required for T cell mediated lung inflammatory responses. However, ICOS on T cells is a co-stimulatory molecule for T cell receptor, while ILC2s lack TCR and there is no evidence suggesting that ILC2s engage any antigen presentation activities. To identify whether function and/or survival of ILC2s requires ICOS the Inventors evaluated viability and cytokine production by pulmonary ILC2s in ICOS^{-/-} and WT mice. Interestingly, the Inventors found that the relative frequency and number of ILC2s are substantially lower in ICOS^{-/-} than in WT mice.

Reduced number of ILC2s in ICOS^{-/-} mice may suggest that ICOS plays an important role in survival and/or proliferation of ILC2s. Analyzing ILC2s apoptosis and cell death the Inventors found that the frequency of dead cells is increased in the absence of ICOS while the frequency of early and late apoptotic ILC2s are comparable. Evaluating ILC2s' proliferation by measuring the expression of Ki-67, a protein that has been associated with cell proliferation, revealed that the rate of proliferation of ILC2s is similar in ICOS deficient and WT mice. These findings suggest that while ICOS provides a survival signal for ILC2s, it seems to be redundant for ILC2s' proliferation.

ICOS substantially contributes to cytokine production by ILC2s. Using intracellular staining and gating on the live cells, the Inventors found that upon stimulation with IL-33, production of IL-13 is significantly reduced in the absence ICOS. Similarly, it has been shown that ICOS plays an important role of the production of IL-13 and IL-4 in T cells. Interestingly, the Inventors found that the level of IL-13 as well as IL-5 in the supernatant of purified ILC2s was significantly lower in ICOS^{-/-} mice than in WT mice. The difference between the results of intracellular cytokine measurement by flow cytometry and measurement of released cytokine in the supernatant by ELISA can be explained by the fact that intracellular staining determines the produced cytokine in live cells however, the supernatant of cell culture reflects the total cytokine that were produced by the seeded cells. Since, the Inventors show that ICOS^{-/-} ILC2s have higher rate of death and given the equal number of seeded cells of ICOS^{-/-} and WT, the total number of cytokine producing cells are lower in ICOS^{-/-} cultures than in WT which explains the lower production of IL-5 in the supernatant of cultured ICOS^{-/-} ILC2s.

Taken together, the Inventors' results suggest that while IL-13 production is impaired in ICOS^{-/-} ILC2s, because of higher rate of death in ICOS^{-/-} ILC2s total production of IL-5 by ILC2s is also reduced in the absence of ICOS. In line with the Inventors' in vitro findings, the Inventors' in vivo results that show a lower number of eosinophils, that are dependent of IL-5 production, in the absence of ICOS or in the presence of blocking anti-ICOS antibody suggest similar findings.

The Inventors show that mouse and human ILC2s express functional ICOS-Ligand and that ICOS:ICOS-Ligand interaction is required for ILC2s' survival and cytokine production. Since the Inventors found that ICOS plays an important role in cytokine production in purified in vitro culture of ILC2s the Inventors evaluated the expression of ICOS-Ligand by ILC2 and found that while WT ILC2s express ICOS-Ligand a low level, ICOS deficient ILC2s express high levels of ICOS-Ligand.

Interestingly, blocking ICOS:ICOS-Ligand interaction antibody increases the expression of ICOS-Ligand by ILC2s. Moreover, blocking ICOS:ICOS-Ligand interaction results in reduced AHR, airway inflammation and lower number of eosinophils and ILC2s in the lungs. These findings suggest that ICOS-Ligand is down-regulated upon binding to ICOS. In agreement with the Inventors' observations, it has previously been shown that ICOS:ICOS-Ligand interaction leads to down-regulation of ICOS-Ligand in B cells. To the Inventors' knowledge this is the first report of expression of ICOS and ICOS-Ligand by the same type of cells. Since it has been reported that ILC2s (previously reported as nuocytes) may express MHC-I and the Inventors observed that they express ICOS-Ligand, whether this cells engage in antigen presentation activities remains to be investigated.

The Inventors' data show that GATA-3 is not affected by lack of ICOS:ICOSL signaling. Several reports have shown that GATA-3 is expressed by ILC2s and plays a crucial role in development, maintenance and function of ILC2s. Moreover, IL-13 production by ILC2s has been associated with high level of expression of GATA-3. However, when the Inventors compared the level of expression of GATA-3 in WT with ICOS^{-/-} ILC2s at steady state and after IL-33 stimulation, the Inventors found comparable level of GATA-3 suggesting that impairment of IL-13 production by ICOS^{-/-} ILC2s is caused by a mechanism other than the reduction of GATA-3 expression.

The Inventors show that STAT-5 signaling is impaired in ICOS^{-/-} ILC2s. Investigating the mechanisms by which ICOS contributes to ILC2 survival and function the Inventors found that the level of phospho-STAT5 in ICOS^{-/-} ILC2s is substantially lower than in WT ILC2s. This finding suggests that lack of ICOS leads to alteration in IL-2 signaling. Interestingly, STAT5 is not only required for IL-2 mediated cell survival and Th2 differentiation but it is also required for efficient production of IL-13 in T cells and mast cells. The Inventors further investigated and show that additional signaling through ICOS by using ICOS-Ligand-FC, that consists of mouse ICOS-Ligand fused with FC part of human IgG, increases the level of phosphor-STAT5 and leads to higher production of IL-13. Taken together, the Inventors' data suggest that ICOS plays an important role in the survival and ILC2s' cytokine production through IL-2/STAT-5 signaling pathway.

The Inventors further provide evidence that ICOS:ICOS-Ligand interaction is not only required for IL-33-mediated ILC2s function and survival but it is also required for the ILC2s-mediated induction of AHR and lung inflammation by a clinically relevant allergen. Alternaria is an abundantly found fungus in the environment and an allergen in humans and has been shown to cause induce allergic inflammation in mice independent of adaptive immunity.Using a similar approach the Inventors found that blocking ICOS:ICOS-Ligand interaction reduces Alternaria-induced AHR and lung inflammation in RAG2^{-/-} mice which confirms the importance of this signaling pathway for efficient function of ILC2s after activation by a clinically relevant allergen.

The Inventors introduce a humanized mouse model, in which human peripheral ILC2s are adoptively transferred to RAG−/− Il2rg^{-/-} mice and i.n. administration of IL-33 causes AHR and inflammation. This mouse model provides a unique platform for studying the contribution of ILC2s to human asthma and evaluating the efficacy of potential therapeutic targets in preclinical studies. Using this model the Inventors show that ICOS:ICOS-Ligand interaction is required for efficient function of human ILC2s in vivo. These findings underscore the importance of ICOS signaling pathway for efficient function of human ILC2s and demonstrate that the Inventors' finding in mouse ILC2s are of high importance for and translatable to clinical studies.

In conclusion, the Inventors' study demonstrates that human and mouse ILC2s express ICOS and ICOS-Ligand and that ICOS:ICOS-Ligand interaction provides a survival signal for ILC2s and is required for efficient cytokine production by ILC2s. The Inventors show that STAT5 signaling pathway is impaired in ICOS^{-/-} ILC2s while it is enhanced through ICOS signaling leading to higher cytokine production. This may provide an explanation for the lower rate of survival and cytokine production in the absence of ICOS or blockade of ICOS:ICOS-Ligand signaling. The Inventors introduce a humanized mouse model where human ILC2s derive AHR, a cardinal feature of asthma in mice and using this system the Inventors show that ICOS:ICOS-Ligand interaction is required for optimal function of human ILC2s in vivo. Since ILC2s are the only cells that express ICOS and ICOS-Ligand, the Inventors' findings set the stage for designing new therapeutic approaches for asthma where ILC2s can be targeted, for instance by dual specific antibodies that recognize ICOS and ICOS-Ligand.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are methods and compositions related to modulating the innate lymphoid cells (ILCs), and related properties via Inducible T-cell COStimulator (ICOS) or ICOS-ligand mediated pathways, method of isolating, characterizing or altering ILCs, or methods and compositions related to ICOS or ICOS-ligand pathways in the treatment of lung and/or airways related tissues, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for treating inflammation in lung and/or airway tissue in a subject, comprising:
selecting a subject with inflammation in lung and/or airway tissue; and
administering a quantity of a therapeutic agent, wherein the therapeutic agent treats inflammation in lung and/or airway tissue.

2. The method of claim 1, wherein the therapeutic agent comprises an antibody capable of binding to ICOS, ICOS ligand, or both, and a pharmaceutically acceptable carrier.

3. The method of claim 1, wherein the therapeutic agent comprises a composition capable of modulating ICOS expression and a pharmaceutically acceptable carrier.

4. The method of claim 1, wherein the therapeutic agent comprises a composition capable of modulating ICOS ligand expression and a pharmaceutically acceptable carrier.

5. The method of claim 1, wherein the therapeutic agent comprises a composition capable of modulating a type 2 inflammatory response, and a pharmaceutically acceptable carrier.

6. The method of claim 5, wherein modulating a type 2 inflammatory response, comprises a reduction in the expression of IL-33 and/or IL-25.

7. The method of claim 5, wherein modulating a type 2 inflammatory response, comprises a reduction in the expression of one or more of: IL-4, IL-5, and IL-13.

8. The method of claim 1, wherein the lung and/or airway tissue comprises bronchiolar and/or aveolar tissue.

9. The method of claim 1, wherein the lung and/or airway tissue comprises epithelial tissue.

10. The method of claim 1, treating inflammation comprises a reduction in the number of innate lymphoid cells (ILCs).

11. The method of claim 10, wherein the ILC are type 2 ILCs (ILC2) cells.

12. The method of claim 11, wherein the ILC2 cells do not express one or more of: CD3, CD14, CD16, CD19, CD20, CD56, CD235a, CD1a, and CD123.

13. The method of claim 11, wherein the ILC2 cells express one or more of: CD45, CRTH2, CD127 and CD161

14. The method of claim 1, wherein treating inflammation comprises a reduction in STAT5 pathway activation in ILCs.

15. A pharmaceutical composition, comprising:
a quantity of a therapeutic agent comprising a composition capable of binding or modulating expression of ICOS, ICOS ligand, or both; and
a pharmaceutically acceptable carrier.

16. The composition of claim 15, wherein the composition comprises an antibody.

17. A method of modulating inflammation, comprising:
selecting a subject in need of treatment for inflammatory related disease and/or condition; and administering a therapeutic agent to the subject, wherein the administration of the composition modulates inflammation in the subject.

18. The method of claim 17, wherein the inflammatory related disease and/or condition is acute.

19. The method of claim 17, wherein the inflammatory related disease and/or condition is chronic.

20. The method of claim 17, wherein the inflammatory related disease and/or condition is a lung related disease and/or condition.

21. The method of claim 17, wherein modulating inflammation in the subject comprises decreased type 2 ILCs (ILC2) cell phenotype.

22. The method of claim 21, wherein the ILC2 cells do not express one or more of: CD3, CD14, CD16, CD19, CD20, CD56, CD235a, CD1a, and CD123.

23. The method of claim 21, wherein the ILC2 cells express one or more of: CD45, CRTH2, CD127 and CD161.
